(19) United States
(12) Patent Application Publication
(10) Pub. No.: US 2026/0140116 A1
(43) Pub. Date: May 21, 2026

(54) METHODS, DEVICES, AND SYSTEMS FOR PREDICTING RESPONSIVENESS OF PROSTATE CANCER PATIENTS TO NEOADJUVANT THERAPY

(71) Applicant: THE REGENTS OF THE UNIVERSITY OF CALIFORNIA, Oakland, CA (US)

(72) Inventors: Hang CHANG, Moraga, CA (US); April W. MAO, Berkeley, CA (US); Jian-Hua MAO, Moraga, CA (US)

(21) Appl. No.: 19/390,270

(22) Filed: Nov. 14, 2025

Related U.S. Application Data
(60) Provisional application No. 63/721,162, filed on Nov. 15, 2024.

(51) Int. Cl. G01N 33/574; G16H 20/40; G16H 50/20

(52) U.S. Cl. CPC G01N 33/57555; G16H 20/40; G16H 50/20

(57) ABSTRACT

The present invention provides for a method for predicting whether a subject with prostate cancer will be responsive to neoadjuvant therapy (NT), or whether NT will be efficacious for treating a subject with prostate cancer. The present invention also provides for a system using machine learning for determining whether a subject with prostate cancer will be responsive to neoadjuvant therapy (NT), or whether NT will be efficacious for treating a subject with prostate cancer.

### CROSS-REFERENCE TO RELATED APPLICATIONS

This application claims priority to U.S. Provisional Patent Application Ser. No. 63/721,162, filed Nov. 15, 2024, which are hereby incorporated by reference.

### STATEMENT OF GOVERNMENTAL SUPPORT

The invention described and claimed herein was made utilizing funds supplied by the U.S. Department of Energy under Contract No. DE-AC02-05CH11231, U.S. Department of Defense (DOD) under Grant BCRP, No. BC190820, and the National Institutes of Health (NIH) under No. R01CA184476. The government has certain rights in this invention.

### FIELD OF THE INVENTION

The present invention is in the field of prostate cancer.

### BACKGROUND OF THE INVENTION

Prostate cancer (PCa) is the most commonly diagnosed non-skin cancer in men in the United States and will be the second-leading cause of cancer-related death in men in 2024 (1), with an estimated 299,010 new cases and 35,250 deaths this year (2). Developing accurate and cost-effective biomarkers to identify men at the greatest risk of poor outcomes following intervention is crucial.

Many patients with localized PCa, i.e., without metastases in other organs or non-regional lymph nodes, can be cured with appropriate management. Depending on the risk stratification using clinical and pathological features (3) and life expectancy, those with low-risk disease may be managed through active surveillance, radical prostatectomy, or radiation therapy (4). In contrast, those with intermediate-high risk may need additional therapies such as androgen deprivation therapy (ADT), androgen receptor signaling inhibitors, and chemotherapy.

Although the effectiveness of neoadjuvant ADT (NADT) was demonstrated in a series of phase-II clinical trials (5-14), both European and American guidelines recommend against the use of NADT prior to radical prostatectomy due to a lack of clinically significant efficacy results and notable side effects (3,15). Other outcomes, such as pathologic complete response rate, varied across cohorts. Correlative studies from window of opportunity trials, however, did show a subgroup of patients that had favorable pathologic responses, and reduced cancer volume at time of surgery (16-18), with the use of NADT, thus suggesting that patients who responded to, or were resistant to, hormonal therapies could be predicted.

### SUMMARY OF THE INVENTION

The present invention provides for a method for predicting whether a subject with prostate cancer will be responsive to neoadjuvant therapy (NT), or whether NT will be efficacious for treating a subject with prostate cancer. The present invention also provides for a system using machine learning for determining whether a subject with prostate cancer will be responsive to neoadjuvant therapy (NT), or whether NT will be efficacious for treating a subject with prostate cancer.

The present invention provides for a method for predicting whether a subject with prostate cancer will be responsive to neoadjuvant therapy (NT), or whether NT will be efficacious for treating a subject with prostate cancer, comprising: (a) obtaining a prostate cancer tumor sample from a subject suffering from prostate cancer, or suspected thereof; (b) determining or measuring the value or quantity of one or more cellular morphometric biomarkers (CMB); (c) correlating the value or quantity determined or measured for the CMBs to determine whether the subject would benefit from a neoadjuvant androgen deprivation therapy (NT); and (d) optionally treating the subject with NT.

The present invention provides for a method for predicting whether a subject with prostate cancer will be responsive to neoadjuvant therapy (NT), or whether NT will be efficacious for treating a subject with prostate cancer, comprising: (a) obtaining a prostate cancer tumor sample from a subject suffering from prostate cancer, or suspected thereof; (b) determining or measuring the value or quantity of one or more cellular morphometric biomarkers (CMB); (c) calculating a Cellular Morphometric Biomarker Risk Score (CMBRS) using the CMBs determined or measured in step (b) using the formula:

$$CMBRS = {Σ}_{i = 1}^{N} ( coefficient of {CMB\_Category}^{i} ) * ( {CMB\_Category}^{i} ) ;$$

(d) correlating the CMRS to whether the subject would benefit from a neoadjuvant therapy (NT); and (d) optionally treating the subject with NT.

In some embodiments, the CMBRS is calculated using the means described herein. In some embodiments, the CMBRS is calculated using the 13-CMB model described herein. CMBs are either favorable to NAPT treatment response, or not favorable to NADT treatment response. The term “unfavorable CMB” suggests that the presence of the CMB means less likely that NADT treatment should be efficacious. The term “favorable CMB” suggests that the presence of the CMB means more likely that NADT treatment should be efficacious. CMB 141 is an unfavorable CMB for NADT treatment response. CMB 141 comprises the presence of benign prostatic acinus (gland) with multilayering due to basal cell hyperplasia. CMBs 212 and 236, are favorable CMBs for NADT treatment response. In some embodiments, the CMBs comprise the CMBs described in Table 1 of Example 1 herein. In some embodiments, the determining or measuring step comprises the value or quantity of the 13 CMBs described in Table 1 of Example 1.

The present invention provides for a system using machine learning for determining the CMRS, which is described herein.

A neoadjuvant therapy (NT) is a cancer treatment that involves giving a patient treatment before the main treatment for the cancer. The goal of neoadjuvant therapy is to make the main treatment more likely to be successful. In some embodiments, the NT comprises a neoadjuvant androgen deprivation therapy (NADT). In some embodiments, the NADT comprises administering an androgen receptor blocker to the subject. In some embodiments, the NT comprises administering an mTOR inhibitor to the subject, or a DNA damage response (DDR) inhibitor to the subject.

Suitable androgen receptor blocker include, but are not limited to, a nonsteroidal antiandrogen that blocks androgen receptors, a second-generation nonsteroidal antiandrogen that blocks androgen receptors, a steroidal antiandrogen that blocks androgen receptors, a CYP17A1 inhibitor that blocks testosterone synthesis in the prostate and adrenal glands, a LURH agonist that suppresses the hypothalamic-pituitary-gonadal axis, or a gonadotropin-releasing hormone (GnRH) antagonist. In some embodiments, the nonsteroidal antiandrogen that blocks androgen receptors is Flutamide (Eulexin), Nilutamide (Nilandron), or Bicalutamide (Casodex). In some embodiments, the second-generation nonsteroidal antiandrogen that blocks androgen receptors is Enzalutamide (Xtandi), Apalutamide (Erleada), or Darolutamide (Nubega). In some embodiments, the steroidal antiandrogen that blocks androgen receptors is Cyproterone acetate. In some embodiments, the CYP17A1 inhibitor that blocks testosterone synthesis in the prostate and adrenal glands is Abiraterone. In some embodiments, the LHRH agonist that suppresses the hypothalamic-pituitary-gonadal axis is Leuprolide Acetate, Triptorelin Pamoate, Goserelin Acetate, or Histrelin Acetate. In some embodiments, the gonadotropin-releasing hormone (GnRH) antagonist is Degarelix.

Suitable mTOR inhibitors include, but are not limited to, Sirolimus (an immunosuppressant used to treat lymphangioleiomyomatosis, prevent organ transplant rejections, and treat perivascular epithelioid cell tumors, Everolimus (kinase inhibitor used to treat various types of malignancies), Temsirolimus (an antineoplastic agent used to treat renal cell carcinoma (RCC)), NVP-BEZ235 (dactolisib) (a generation DI compound developed by Novartis), OSI-027 (a potent and selective dual inhibitor of mTORC1 and mTORC2), Torin 1 (developed by AstraZeneca with a low nanomolar IC50 against mTOR), Ku-0063794 (an ATP-competitive mTOR inhibitor with strong anti-proliferative activity against cancer cells), AZD8055 (an orally accessible version of Ku-0063794 with antiproliferative action), and XL388 (a selective small-molecule ATP-competitive mTOR inhibitor that inhibits mTORC1 and mTORC2).

Suitable DNA damage response (DDR) inhibitors include, but are not limited to, a PARP inhibitor, a CHK1 inhibitor, an ATR inhibitor, AZD 1390, Prexasertib, SRA 737, Peposertib, Talazoparib, and ZN-c3. PARP inhibitors (such as, Rucaparib (Clovis), olaparib (AstraZeneca), and niraparib (Tesaro) are some of the first DDR inhibitors approved to treat cancer. PARP inhibitors can be used to improve the effectiveness of chemotherapy and radiotherapy. GDC-0575, LY3300054, MK-8776 (SCH-900776), and Prexasertib (LY2606368) are some examples of CHK1 inhibitors. Ceralasertib (AZD6738), BAY1895344, Berzosertib (M6620, VX-970), and M4344 (VX-803) are some examples of ATR inhibitors. AZD 1390 is an ATM protein inhibitor that is in phase I trials for GBM, NSCLC, and soft tissue sarcoma. Prexasertib is a CHEK1 and CHEK2 inhibitor that is in phase II trials for OC and solid tumors. SRA 737 is a CHEK1 inhibitor that is in phase II trials for OC, PC, and solid tumors. Peposertib is a DNA-PK inhibitor that is in phase I/II trials for RC/SCLC, GBM, and cancer. Talazoparib is a DDR-targeted inhibitor that has been used in clinical trials for lung, BRCA or ATM mutant solid tumors, and metastatic endometrial cancer. ZN-c3 is a WEE1 inhibitor that has been granted fast track designation by the FDA for uterine serous carcinoma.

In some embodiments, the subject is a human patient suffering from, or suspected of suffering from, prostate cancer.

With the development of several clinically effective androgen receptor signaling inhibitors for patients with prostate cancer, it is imperative to identify patients who will benefit from these drugs that can impact quality of life functions upon prolonged use. Therefore, it is essential to continue refining predictive models to guide their use. Biomarker discovery is aided by prior clinical trials that evaluated neoadjuvant androgen deprivation therapy (NADT) followed by radical prostatectomy to provide a “window of opportunity” for an extensive evaluation of molecular features of cancer altered by these agents.

Methods: Cellular morphometric biomarker via machine learning, was applied to the extensively validated artificial intelligence technique, on whole slide digital pathology images of specimens from needle biopsies of 37 patients enrolled in a clinical trial (NCT02430480) in which NADT was administered. Predictive modeling using the clinical data and treatment-naïve prostate needle biopsy was designed to predict response to NADT. Validation cohorts consisted of independent specimens and The Cancer Genome Atlas prostate adenocarcinoma (TCGA-PRAD). Immunohistochemical staining was used to quantify the expression profiles of proteins involved in the mTOR pathway.

Results: 13 cellular morphometric biomarkers (CMBs) were identified from whole slide images of needle biopsies from the trial specimens where patients were categorized as exceptional responders (ER) or incomplete or non-responders (INR) based on a residual cancer burden cut-point (0.05 cm³) on pathology. These were used to construct a 13-CMB model that accurately predicted response to NADT (AUC: 0.980; accuracy: 0.892; sensitivity: 0.818; specificity: 1.000). Notably, the 13-CMB model was able to stratify prostate cancer patients after NADT treatment in an independent hospital cohort (n=122) into ER-like and INR-like groups that significantly associated with pathologic complete response (p=0.0005) and biochemical-recurrence-free survival (p=0.024). Genetic analysis revealed interplay between four single nucleotide polymorphisms and two CMBs that may contribute to NADT resistance. And genomic analysis provided the underlying molecular annotations in the TCGA-PRAD cohort. The four genes associated single nucleotide polymorphisms were significant and independent predictors of outcome in the TCGA-PRAD cohort, and their combination with CMBs further improved the prognostic power of this model. In addition, the significantly stronger activation of mTOR signaling pathway was discovered in the TCGA-PRAD INR-like group using the corresponding proteomics data (p70S6K, p=1.6e-5) and validated in the INR-like group in the independent hospital cohort (p70S6K, p=0.03), which led to significantly higher sensitivity to mTOR inhibitor (Rapamycin, p=8.3e-5) and potentially more clinical benefit from mTOR inhibitor in the INR-like group.

Conclusion: The clinical use of CMBs to identify patients likely to benefit from NADT as well as those likely to have better prognosis was evaluated and validated. The findings uncover the complex interplay between genetic variants, and cellular morphometric architecture that may contribute to NADT resistance; and identified patients likely to benefit from mTOR inhibitor. CMBs have predictive value for precision treatment and management of prostate cancer patients, which warrants evaluation in larger multicenter cohorts.

In some embodiments, the method comprises: (1) AI-Powered cellular morphometric biomarkers discovered in needle biopsy of prostatic cancer predict neoadjuvant androgen deprivation therapy response and prognosis; (2) AI-Powered cellular morphometric biomarkers discovered in needle biopsy of prostatic cancer predict potential benefit from mTOR inhibitors; (3) biomarkers developed from clinical trial data and validated in independent hospital cohorts; (4) potential benefit from mTOR inhibitors validated using immunohistochemical (IHC) staining.

Other objects, features, and advantages of the present invention will be apparent to one of skill in the art from the following detailed description and figures.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1. (A-D) Study design. (A) CMB-ML pipeline for the discovery of CMBs from needle biopsy of NADT patients; (B) Discovery cohort; (C) hospital validation cohort; (D) molecular annotation. (E) Representative examples of CMBs. (F) Association of individual CMB with treatment resistance; (G) Predictive probability between ER and INR groups across different models, where the 4-factor model is based on IDC (presence of intraductal carcinoma); 10q loss (at least half of chromosome arm 10q deleted hemizygously as determined using the GISTIC algorithm); ERG (overexpression of nuclear ERG determined by immunohistochemistry); TP53 (loss-of-function alterations or hotspot mutations to TP53, including copy number loss, as determined by GISTIC); (H) Receiver operating characteristic (ROC) curve across different models. The p values in (F) were obtained using logistic regression, and the p values in (G) were obtained using Non-parametric Mann-Whitney tests. Abbreviations: ER: Exceptional responder; INR: Incomplete and non-responder. Panels (A-D) are created with BioRender.com.

FIG. 2. (A) Patient inclusion chart for the hospital validation cohort. (B) 13-CMB model is significantly associated with NADT response. (C) 13-CMB model is significantly associated with biochemical recurrence-free survival. (D-F) 13-CMB model stratifies TCGA-PRAD patients into groups with significantly different resistance risk score (D) and progression-free survival (E) but no difference in overall survival (F). (G-I) Re-optimization of 13 CMBs in the TCGA-PRAD cohort provides independent and significant prognostic value after adjusting for clinical factors (G) that stratify TCGA-PRAD patients into groups with significantly different progression-free survival (H) despite no difference in overall survival (I). The p-value in (B) was obtained using a Chi-square test, the p values in (C, E, F, H, I) were obtained using log-rank tests, the p value in (D) was obtained using a non-parametric Mann-Whitney test, the p value in (G) was obtained using a multivariate Cox Proportional-Hazards model.

FIG. 3. (A) Association network between CMBs and risk SNPs previously identified by GWAS projects as having significant associations with prostate cancer; (B) Four Genetic variants and two CMBs that mediate NADT treatment resistance together (ADE: Average Direct Effects; ACME: Average Causal Mediation Effects). FIG. 4, panel B is created with BioRender.com.

FIG. 4. (A-C) Four genes associated with four SNPs (identified by mediation analysis) provide independent and significant prognostic value after adjusting for clinical factors and CMBRS (A). These stratify TCGA-PRAD patients into groups with significantly different progression-free survival after adjusting for clinical factors (B), despite no difference in overall survival (C). The combination of 13-CMB and 4-Gene signatures provides independent and significant prognostic value (D) that stratifies TCGA-PRAD patients into groups with significantly different progression-free survival (E) despite no difference in overall survival (F). P values were obtained using Log-Rank tests.

FIG. 5. Association between ER- or INR-like groups and signatures related to tumor features, tumor microenvironment (TME), and metabolism (A); Examples of significantly different signature scores between ER- or INR-like groups (B, top pane); and prediction of these signature scores on hold-out samples during 5-fold cross-validation (B, bottom pane). P values in (B) were obtained using Spearman correlation.

FIG. 6. Predicted (A) HALLMARK_MTORC1_SIGNALING, (B) NK_cells, (C) EMT, and (D) Homologous_recombination scores before and after NADT treatment in the independent hospital cohort, using CMB-Signature models pre-built from TCGA-PRAD cohort. (E-F) Protein expression of p-mTOR and p70S6K in TCGA-PRAD ER-like and INR-like tumors. (G-H) Sensitivity prediction of mTOR inhibitors (i.e., rapamycin and temsirolimus) between TCGA-PRAD ER-like and INR-like tumors based on RNA-seq data using pRRophetic in TCGA-PRAD cohort, where rapamycin shows significantly higher sensitivity in the INR-like tumors as indicated by lower predicted IC50 values. (I-K) Immunohistochemical (IHC) staining shows the tendency of more p-mTOR in INR-like tumors in the independent hospital cohort, and (L) the p-mTOR protein expression is strongly and significantly correlated with the predicted HALLMARK_MTORC1_SIGNALING score. (M-O) Immunohistochemical (IHC) staining shows significantly more p70S6K in INR-like tumors in the independent hospital cohort, and (P) the p70S6K protein expression is strongly and significantly correlated with the predicted HALLMARK_MTORC1_SIGNALING score. Scale bar=100 μm, p values in (A-H, P, O) were obtained using Non-parametric Mann-Whitney tests, and p values in (L, P) were obtained using Spearman correlation.

FIG. 6. Predicted (A) HALLMARK_MTORC1_SIGNALING, (B) NK_cells, (C) EMT, and (D) Homologous_recombination scores before and after NADT treatment in the independent hospital cohort, using CMB-Signature models pre-built from TCGA-PRAD cohort. (E-F) Protein expression of p-mTOR and p70S6K in TCGA-PRAD ER-like and INR-like tumors. (G-H) Sensitivity prediction of mTOR inhibitors (i.e., rapamycin and temsirolimus) between TCGA-PRAD ER-like and INR-like tumors based on RNA-seq data using pRRophetic in TCGA-PRAD cohort, where rapamycin shows significantly higher sensitivity in the INR-like tumors as indicated by lower predicted IC50 values. (I-K) Immunohistochemical (IHC) staining shows the tendency of more p-mTOR in INR-like tumors in the independent hospital cohort, and (L) the p-mTOR protein expression is strongly and significantly correlated with the predicted HALLMARK_MTORC1_SIGNALING score. (M-O) Immunohistochemical (IHC) staining shows significantly more p70S6K in INR-like tumors in the independent hospital cohort, and (P) the p70S6K protein expression is strongly and significantly correlated with the predicted HALLMARK_MTORC1_SIGNALING score. Scale bar=100 μm, p values in (A-H, P, O) were obtained using Non-parametric Mann-Whitney tests, and p values in (L, P) were obtained using Spearman correlation.

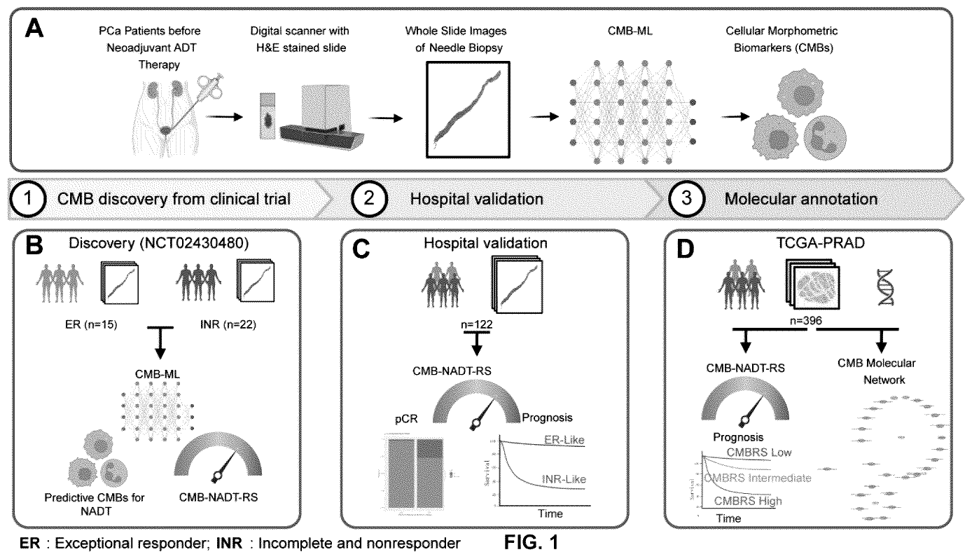

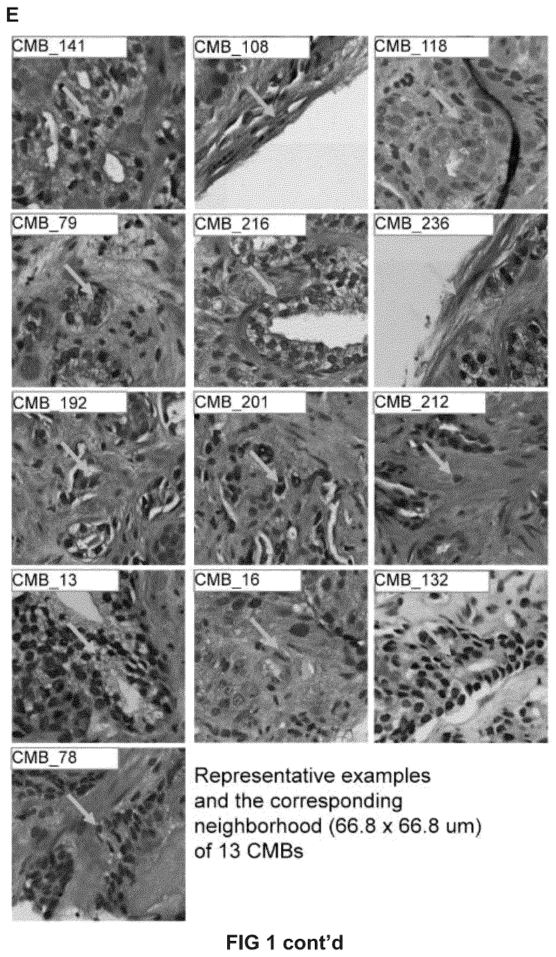

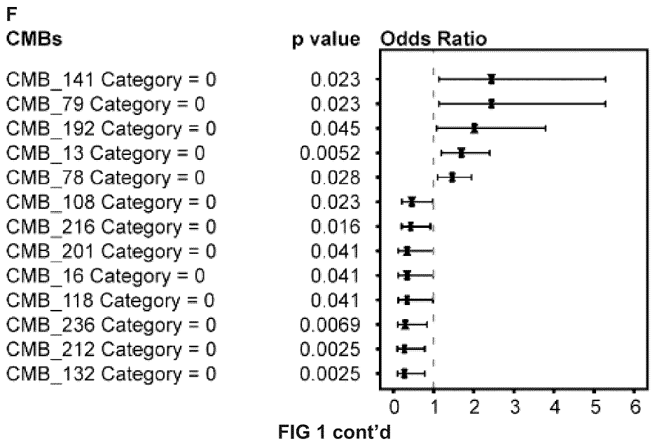

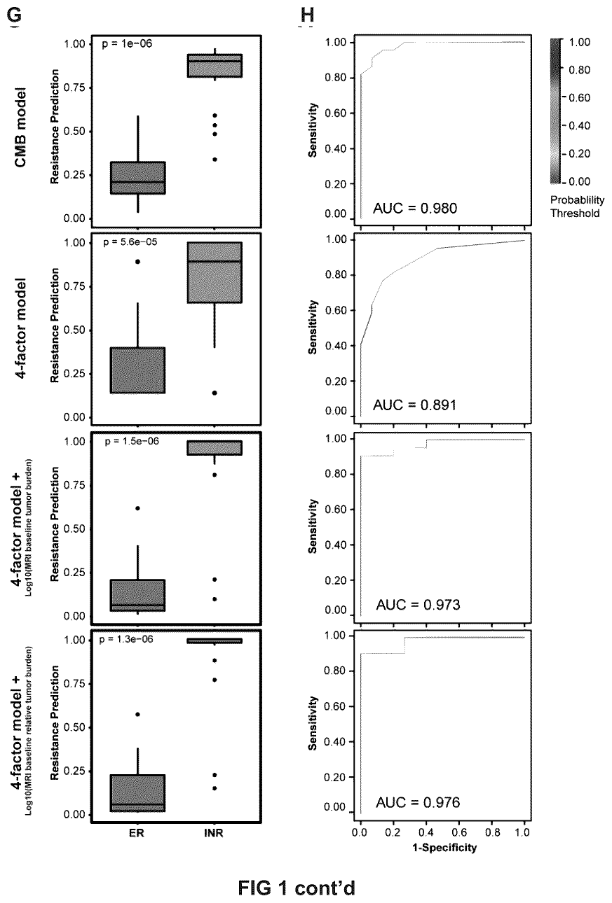

Supplementary FIG. 1. Model construction and threshold optimization. (A) Lambda of LASSO during cross-validation; (B) Coefficient of CMBs in LASSO; (C) Cutoff optimized via bootstrapping on Youden index.

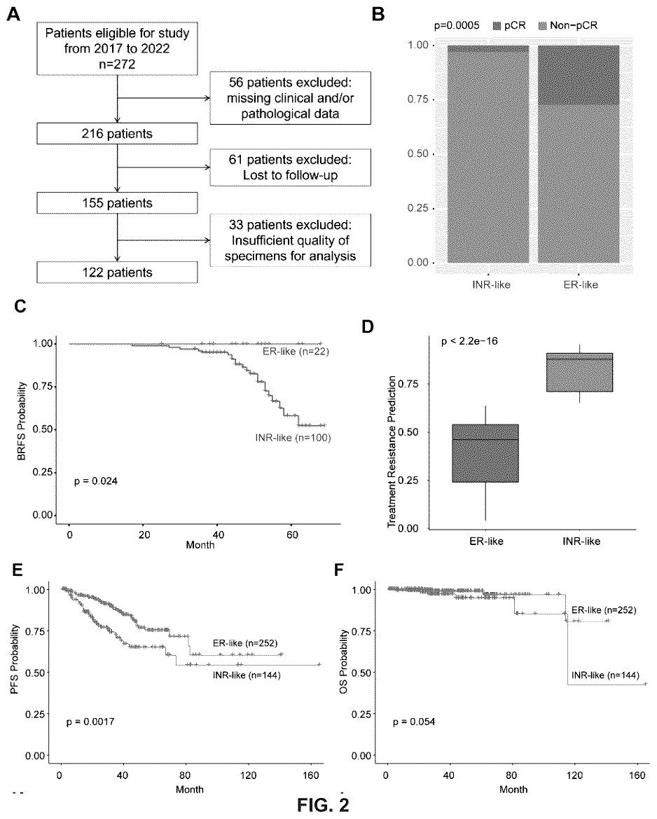

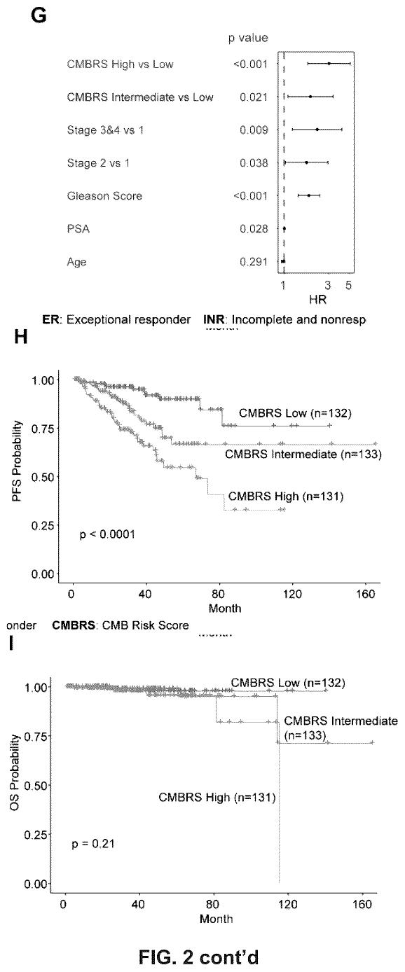

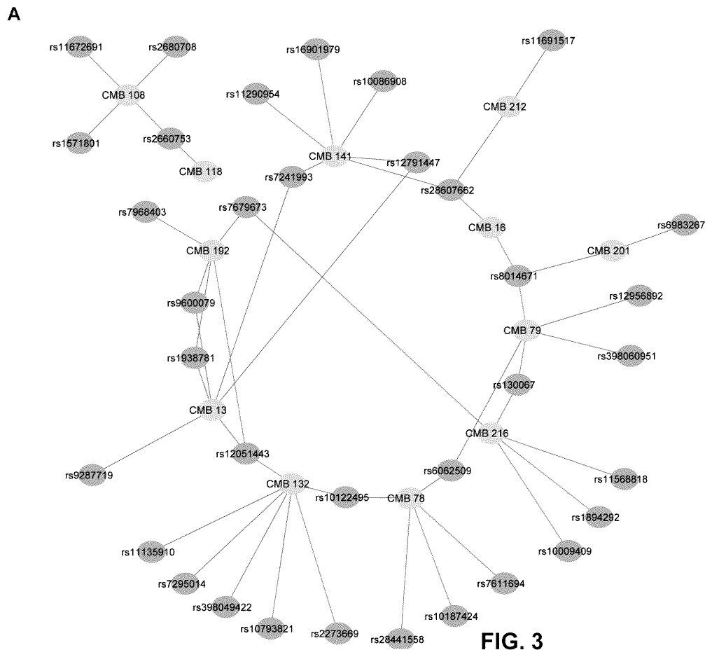

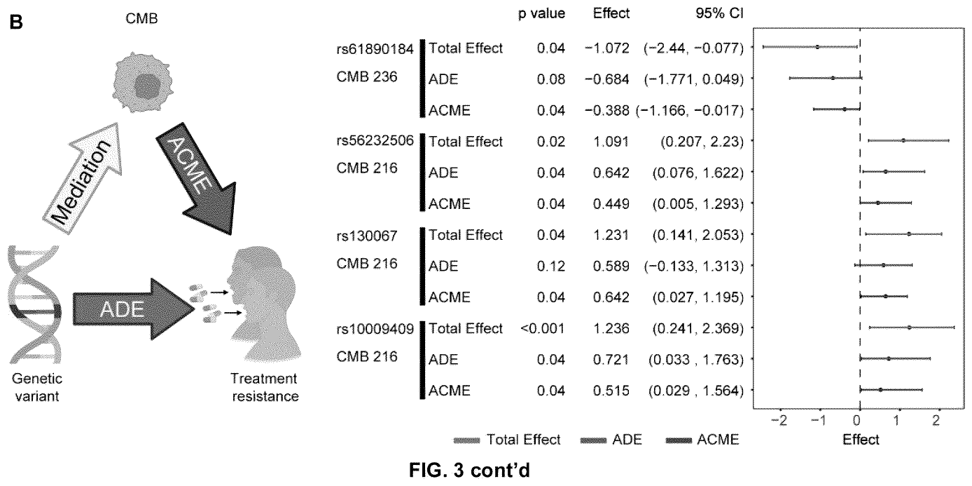

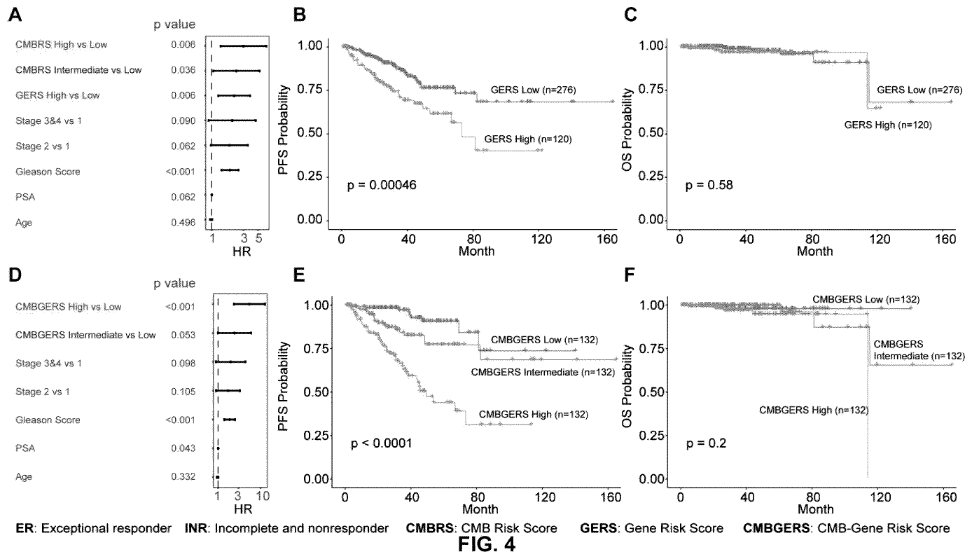

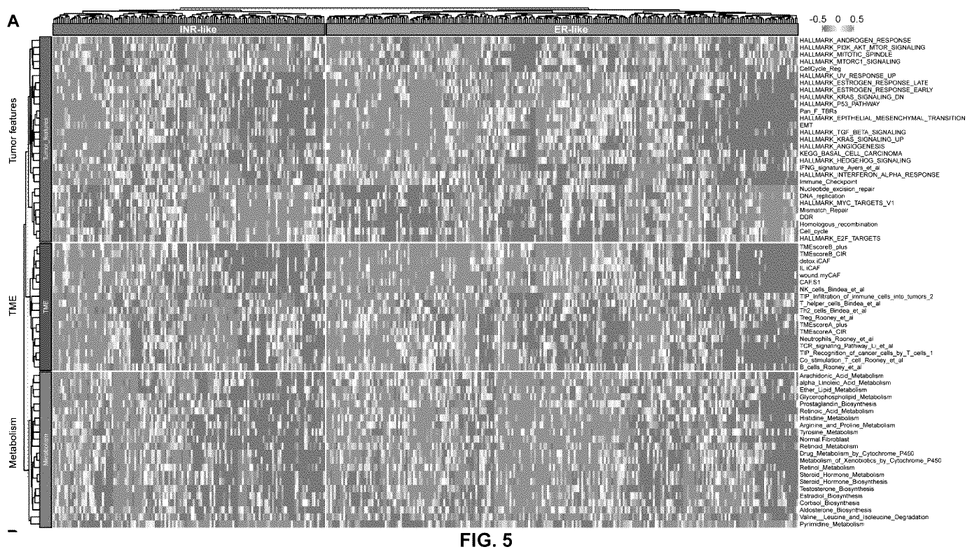

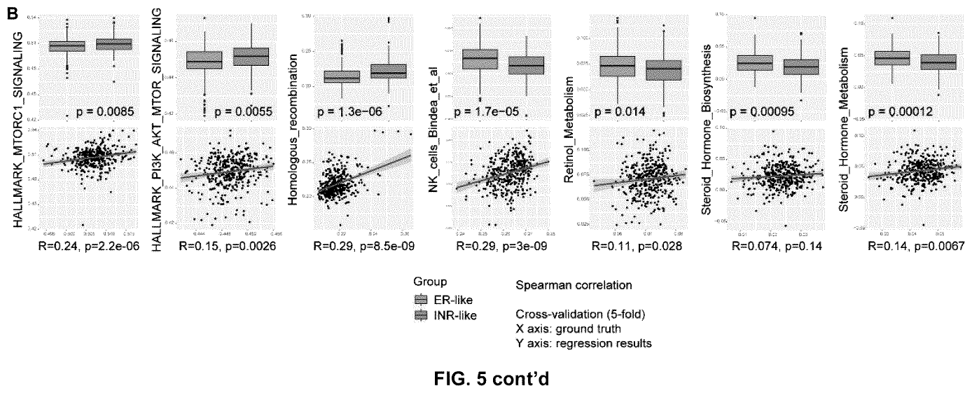

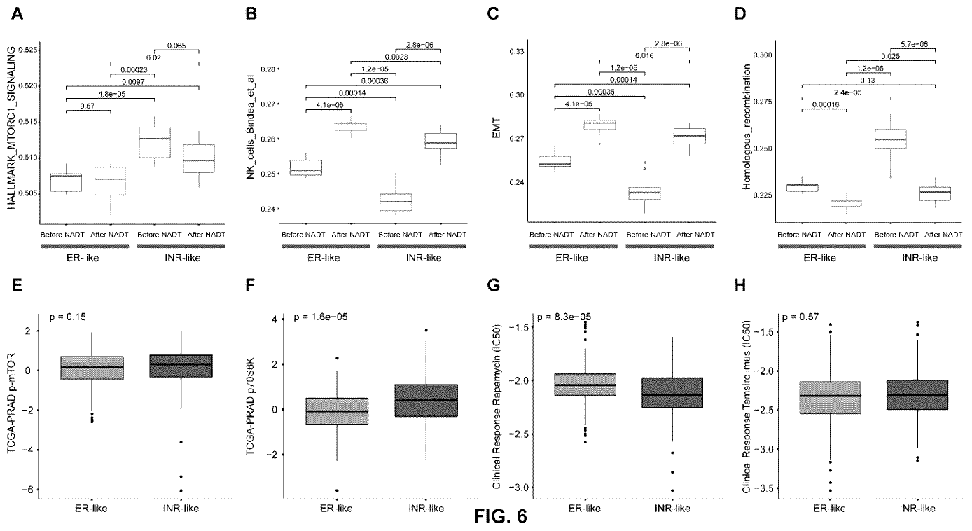

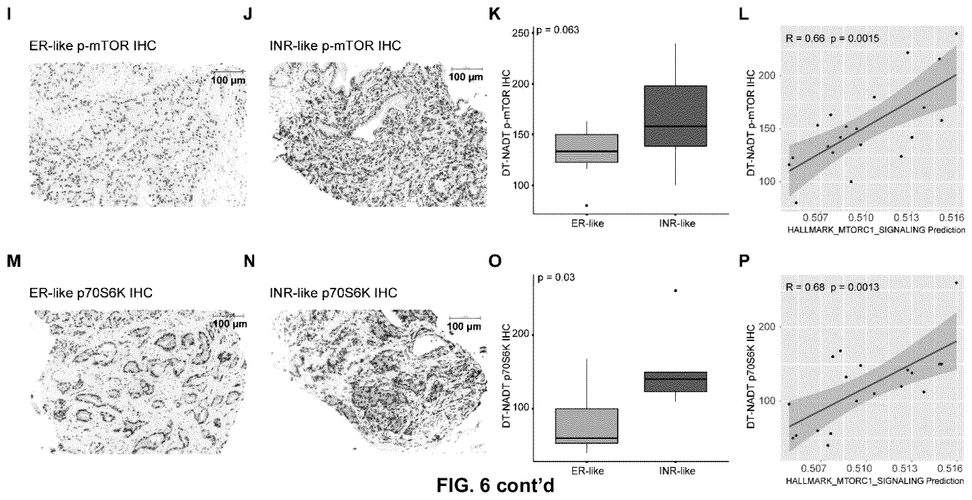

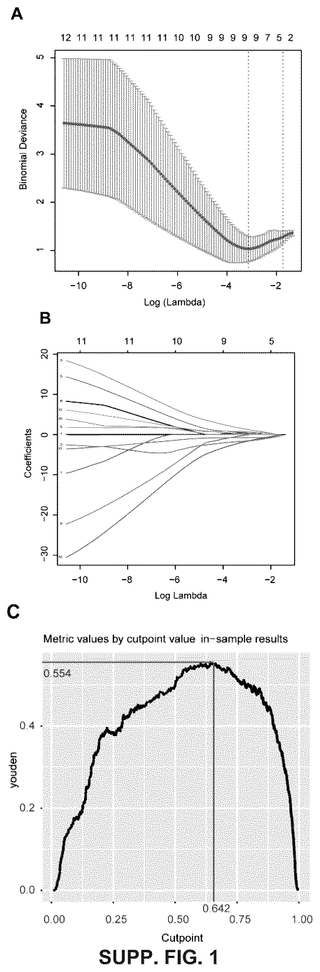

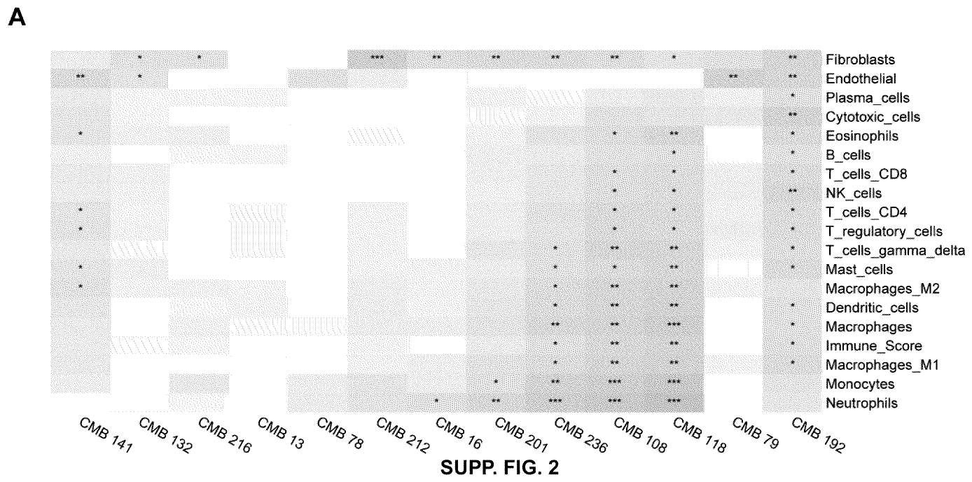

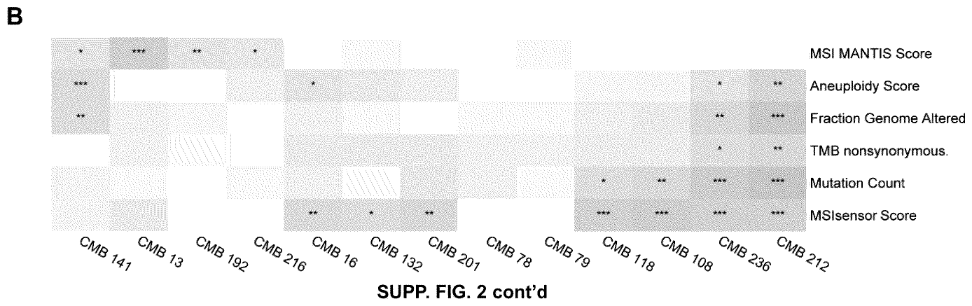

Supplementary FIG. 2. Association between CMBs and tumor microenvironment (A) and genome instability (B). Significance levels were obtained using Spearman correlation (*p<0.05; **p<0.01; ***p<0.001).

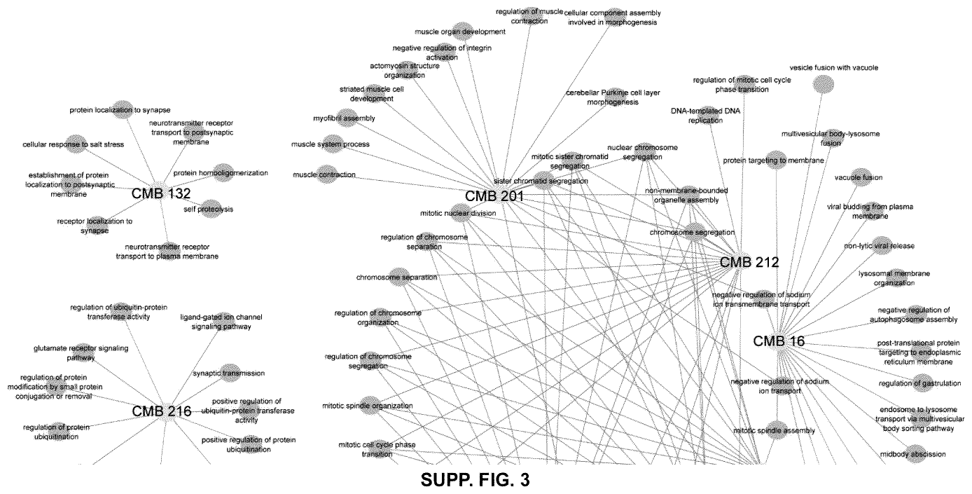

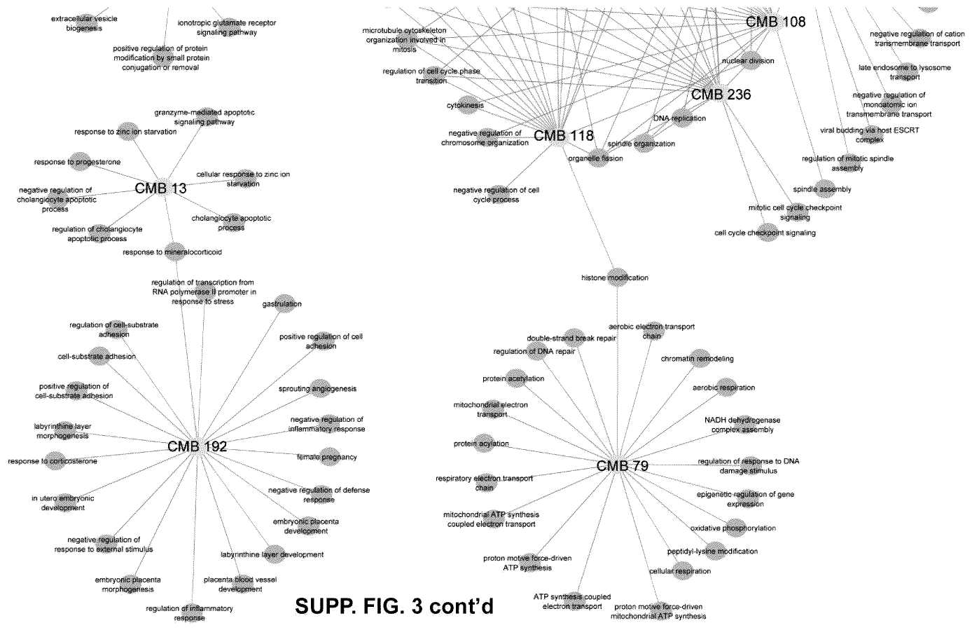

Supplementary FIG. 3. CMB-BP-Network. The network of significantly enriched biological process (BP) on genes correlated with CMB.

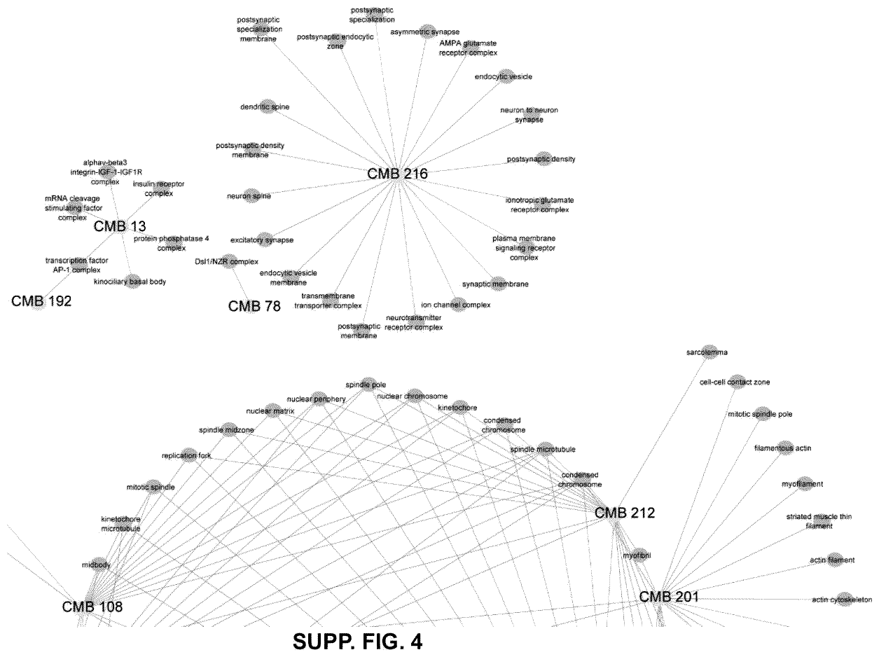

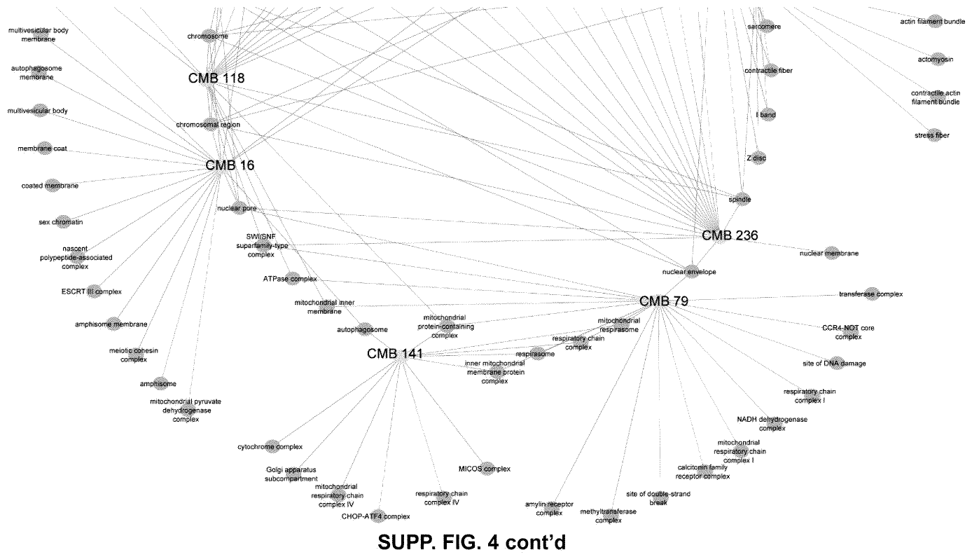

Supplementary FIG. 4. CMB-CC-Network. The network of significantly enriched cellular components (CC) on genes correlated with CMB.

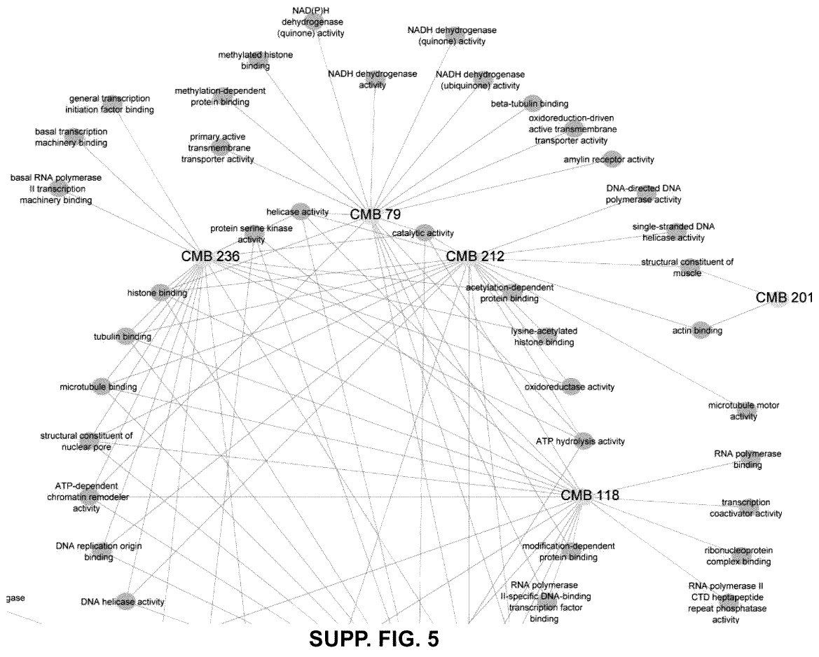

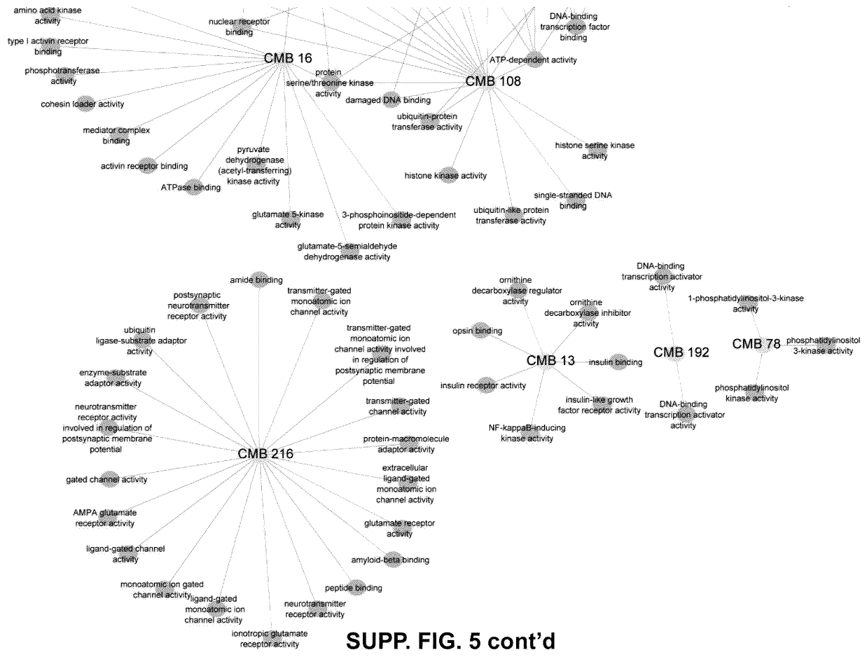

Supplementary FIG. 5. CMB-MF-Network. The network of significantly enriched molecular function (MF) on genes correlated with CMB.

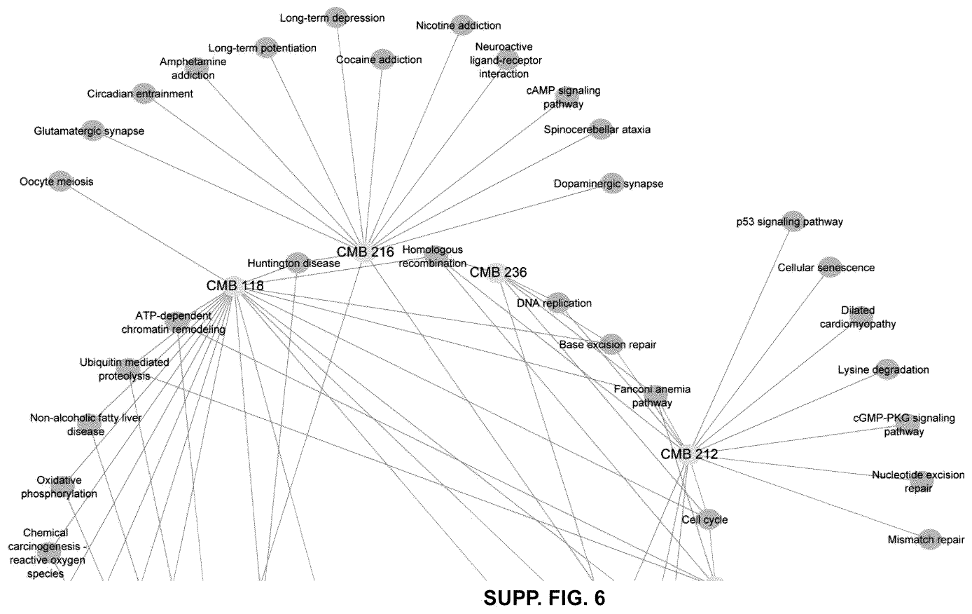

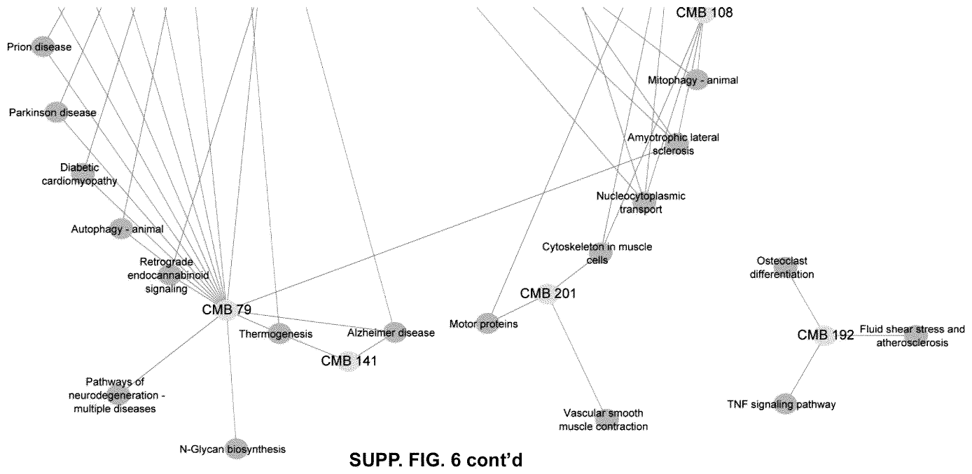

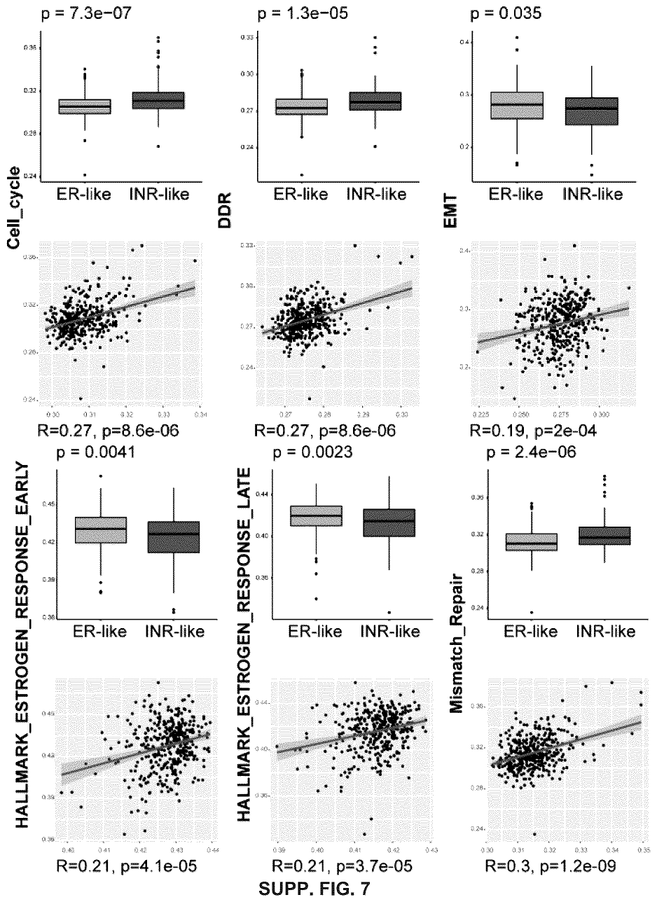

Supplementary FIG. 6. CMB-KEGG-Network. The network of significantly enriched Kyoto Encyclopedia of Genes and Genomes (KEGG) pathway on genes correlated with CMB.

Supplementary FIG. 7. Examples of significantly different signature scores between ER- or INR-like groups (top pane) and prediction of these signature scores on hold-out samples during 5-fold cross-validation (bottom pane). The p values in the top pane were obtained using Non-parametric Mann-Whitney tests, and the p values in the bottom pane were obtained using Spearman correlation.

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

As used in the specification and the appended claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “cell” includes a single cell as well as a plurality of cells; and the like.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

The term “about” as used herein means a value that includes 10% less and 10% more than the value referred to.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

### AI-Powered Cellular Morphometric Biomarkers Discovered in Needle Biopsy of Prostatic Cancer Predict Neoadjuvant Androgen Deprivation Therapy Response and Prognosis: An International Multicenter Retrospective Study

It is imperative to identify patients with prostate cancer (PCa) who will benefit from androgen receptor signaling inhibitors that can impact quality of life upon prolonged use. Using our extensively-validated artificial-intelligence technique: cellular morphometric biomarker via machine learning (CMB-ML), we identified 13 CMBs from whole slide images of needle biopsies from the trial specimens (NCT02430480, n=37) that accurately predicted response to neoadjuvant androgen deprivation therapy (NADT) (AUC: 0.980). Notably, 13-CMB model stratified PCa patients into responder and non-responder groups after NADT treatment in an independent hospital cohort (n=122) that significantly associated with pathologic complete response (p=0.0005), biochemical-recurrence-free survival (p=0.024) and mTOR signaling pathway (p=0.03), suggesting potentially more clinical benefit from mTOR inhibitors in non-responder group. Additionally, genetic and genomic analysis revealed interplay between genetic variants and CMBs on NADT resistance, and provided molecular annotations for CMBs. Overall, prospective clinical implementation of 13-CMB model could assist precision care of PCa patients. (Note that the use of the pronouns “we” may refer to individuals other than the named inventors herein.)

### Significance

We describe a highly accurate CMB model to predict the therapeutic benefit in prostate cancer patients, and uncover the complex interplay between genetic variants and CMBs on NADT resistance. Our model relies only on widely available needle biopsy specimens, and provides a robust and cost-effective solution for clinical implementation.

Multidisciplinary cancer research has been essential for precision medicine and personalized therapy of cancer patients, including patients diagnosed with PCa. To allow accurate and cost-effective cancer patient stratification, we have recently developed and validated an artificial intelligence framework for cellular morphometric biomarker (CMB) discovery from whole slide images (WSIs) of tissue histology. In other cancers, CMBs are associated with specific molecular alterations, immune microenvironment, prognosis, or treatment response (19). In this study, we hypothesized that CMBs assessed from PCa can enable precision prognosis and prediction of response to ADT or other therapeutic agents.

### Results

### Retrospective Study Cohorts

The clinical trial cohort (NCT02430480) enrolled thirty-seven patients with intermediate or high-risk PCa, whose targeted biopsies had been obtained before they received six months of NADT plus enzalutamide. A cut point of 0.05 cm3 for residual cancer burden was used to define exceptional responders (ER, n=15) and incomplete and non-responders (INR, n=22) (20,21). Sections of formalin-fixed, paraffin-embedded (FFPE) needle biopsy tissues were stained with hematoxylin and eosin (H&E), and slides were scanned at 20× magnification. The retrospective validation hospital cohort consisted of 122 needle biopsies (obtained before NADT treatment) from localized primary PCa patients at or above intermediate risk according to National Comprehensive Cancer Network (NCCN) risk classification (22) with complete clinical, pathological, and follow-up information. Sections of H&E stained, FFPE needle biopsy slides were scanned at 20× magnification. The TCGA-PRAD cohort comprises 396 H&E-stained diagnostic slides from localized primary PCa patients with matching clinical data.

### CMBs Accurately Predict NADT Response in the NCT02430480 Trial Cohort

CMBs are artificial intelligence-mined imaging biomarkers from WSIs that have demonstrated association with tumor microenvironments, prognosis, and treatment response in different tumor types (23-25) and model systems (26). The predictive CMBs (FIG. 1, panels A and B) were mined from baseline tissue needle biopsy from patients with intermediate- to high-risk PCa categorized as ER (n=15) or INR (n=22). A total of 256 CMBs were identified, among which the abundance levels of 13 CMBs (i.e., low and high) were significantly associated with treatment response (FIG. 1, panels E and F, chi-square test, p<0.05; Table 1). A LASSO regression model based on the abundance levels of these 13 CMBs (13-CMB model) accurately predicted response to NADT (FIG. 1, panel G, AUC: 0.980; accuracy: 0.892; sensitivity: 0.818; specificity: 1.000), compared to other promising models previously reported (20). Precisely (FIG. 1, panels G and H), the baseline 4-factor-model (AUC=0.891) consists of four factors: IDC-P (presence of intraductal carcinoma); 10q loss (at least half of chromosome arm 10q deleted hemizygously as determined using the GISTIC algorithm (27)); ERG (immunohistochemical overexpression of nuclear ERG); TP53 (loss-of-function alterations or hotspot mutations to TP53, including copy number loss, as determined by GISTIC). The 4-factor model performance was further improved by including magnetic resonance imaging baseline tumor burden (AUC=0.973) or baseline relative tumor burden (AUC=0.976). Despite these refinements on the 4-factor model, the CMB model continued to perform better (FIG. 1, panels G and H) in comparison, thus making the CMB model an accurate option for patient selection.

TABLE 1
Pathological interpretation of representative cellular morphometric
biomarkers (CMBs) as illustrated in FIG. 2, Panel A.

| CMB | Interpretation |
|---|---|
| CMB 13 | Benign prostatic acinus (gland) with multilayering due to |
| | basal cell hyperplasia. Arrow is pointing to a secretory |
| | cell nucleus |
| CMB 16 | Prostatic adenocarcinoma. Arrow is a smooth muscle nucleus |
| CMB 78 | Benign prostatic gland with basal cell hyperplasia. Arrow |
| | is pointing at a basal cell |
| CMB 79 | Prostatic adenocarcinoma, grade 3/low grade |
| CMB 108 | Stroma with arrow pointed at a muscle cell |
| CMB 118 | Prostatic adenocarcinoma, grade 3/low grade |
| CMB 132 | Benign prostatic acinus with basal cell hyperplasia |
| CMB 141 | Prostatic adenocarcinoma, grade 3/low grade |
| CMB 192 | Prostatic adenocarcinoma, grade 3 + 4/intermediate grade |
| CMB 201 | Benign prostatic tissue, stromal cells with degenerative change |
| CMB 212 | Benign prostatic glands and stroma. Arrow is on stromal cell |
| CMB 216 | Benign prostatic acinus (gland) with multilayering due to |
| | basal cell hyperplasia. Arrow is pointing to a secretory |
| | cell nucleus |
| CMB 236 | Benign prostatic glands and stroma. Arrow is on stromal cell |

CMBs Stratified PCa Patients Treated with NADT from an Independent Hospital Cohort into Groups with Distinct Treatment Responses and Prognosis

To test the predictive value of the 13-CNVM model, an independent cohort of 122 localized PCa patients treated with NADT was assessed (FIG. 2, panel A). 13-CNVM model classified patients into ER-like (n=22) and INR-like (n=100) groups. The ER-like group had significantly better rates of pathologic complete response (p=0.0005, FIG. 2, panel B), and significantly better biochemical-recurrence-free survival (p=0.024, FIG. 2, panel C). We next applied the 13-CNVM model on the TCGA-PRAD cohort of 396 localized primary PCa patients with diagnostic slides, and classified them into ER-like (n=252) and INR-like (n=144) groups (FIG. 2, panels D-F). In this cohort, the ER-like group had significantly better progression-free survival (PFS) (FIG. 2, panel E, p=0.001′7) than patients in the INR-like group. Lastly, we constructed a cellular morphometric biomarker risk score (CMBRS) based on 13 CMVBs that stratified the TCGA-PRAD cohort into CMBRS-low, intermediate, and high groups with significant prognostic value after adjusting for important clinical factors such as stage, Gleason score, PSA at the time of diagnosis, and age (FIG. 2, panels G-I). Our findings confirm that the 13-CMB model carries both predictive and prognostic value in patients with localized PCa treated with/without NADT.

### The Interplay Between Genetic Variants and Cellular Morphometric Biomarkers Contributes to NADT Resistance

Germline genetic single nucleotide polymorphisms (SNPs) have been identified by genome-wide association studies and found to be associated with PCa (28) and the treatment response to ADT (29). However, the interplay between genetic variants and cellular morphometric architecture on treatment response remains unknown. To explore the potential relationships between genetic variants and CMBs as well as their synergistic effect on the response to NADT, we studied the 146 risk SNPs profiled in the NCT02430480 cohort. Among the 13 CMBs, 12 were significantly associated (Mann-Whitney non-parametric, p<0.05) with 34 SNPs (FIG. 3, panel A). Interestingly, through mediation analysis, we identified that the interplay between 4 SNPs and 2 CMBs (rs61890184 and CMB 236; rs56232506 and CMB 216; rs130067 and CMB 216; rs10009409 and CMB 216) significantly contribute to the individual response to NADT (total effect p<0.05; average causal mediation effects (ACME) p<0.05; FIG. 3, panel B). Unsurprisingly, the four genes associated with above four SNPs (rs10009409: COX18; rs130067: CCHCR1; rs56232506: TNS3; rs61890184: PPFIBP2) provide significant and independent prognostic value in TCGA-PRAD cohort, after adjusting for CMBs and key clinical factors such as stage, Gleason score, PSA level at diagnosis and age (FIG. 4, panels A-C). The multimodal signature (combining the SNP-associated four genes and 13 CMBs) improved stratification in the TCGA-PRAD cohort, especially for identifying the patient subgroup with the poorest PFS, and hence worse prognosis (FIG. 4, panels D-F). Our findings uncover a complex interaction between genetic variants and cellular morphometric architecture that together help predict response to NADT.

CMBs are Significantly Associated with Tumor Microenvironment

PCa has a distinct tumor microenvironment (TME) consisting of stromal cells, immune cells, and a dense extracellular matrix. The TME has been shown to play a role in determining survival, therapeutic response, and metastasis (30). The TCGA-PRAD cohort enables efficient assessment of the association between CMBs and the previously inferred (i.e., using RNA-seq) TME constituents (Supplementary FIG. 2, panel A). Interestingly, CMB 141, an unfavorable CMB in NADT treatment response (FIG. 1, panel F, Odds Ratio>1), was seen to be significantly and positively associated with immune cell infiltration in the TME, including CD4+ T cells, regulatory T cells, mast cells and M2 macrophages. At the same time, CMB 236, a favorable CMB for NADT treatment response (FIG. 1, panel F, Odds Ratio<1), was seen to be significantly and negatively associated with immune cell infiltration in the TME.

CMBs are Significantly Associated with Genome Instability

As a heterogeneous multifocal cancer, localized PCa has signs of genomic instability (31,32) that are associated with recurrence and progression to aggressive cancer (33). Using the resources provided in the TCGA-PRAD cohort, we revealed a significant association between CMBs and genomic instability (GI) (Supplementary FIG. 2, panel B). Specifically, CMB 141, an unfavorable CMB in NADT treatment response (FIG. 1, panel F, Odds Ratio>1), was significantly associated with elevated genomic instability in terms of aneuploidy score, microsatellite instability (MSI) MANTIS score, and fraction of genome altered. CMB 212 and 236, favorable CMBs for NADT treatment response (FIG. 1, panel F, Odds Ratio<1), were significantly associated with lower genomic instability regarding tumor mutation burden, nonsynonymous mutation count, and MSI sensor score.

CMBs are Significantly Associated with Essential Molecular Functions

To gain insight into molecular annotation underlying CMBs, we identified genes significantly associated with individual CMB in the TCGA-PRAD cohort, performed Gene Ontology (GO) functional enrichment analysis on biological process (BP), cellular components (CC), molecular function (MF), and Kyoto Encyclopedia of Genes and Genomes (KEGG) pathway, and explored the molecular annotation underlying CMBs through the CMB-BP-Network (FIG. 5, panel A), CMB-CC-Network (Supplementary FIG. 3), CMB-MF-Network (Supplementary FIG. 4) and CMB-KEGG-Network (Supplementary FIG. 5). Interestingly, many of the CMBs (e.g., CMB 16, CMB 108, CMB 118, CMB 201, CMB 212, CMB 236) co-registered with cell cycle-related biological processes, which play an essential role in drug resistance such that inhibition of prostate cell proliferation helps overcome the resistance to AR inhibitors (34). Other CMBs are associated with distinct functional groups (e.g., CMB 132, CMB 216), revealing potentially different molecular functions underlying these CMBs.

ER-Like and INR-Like Patients have Distinct Signatures Related to Tumor Features, TME, and Metabolism

In the TCGA-PRAD cohort, the distinction of molecular features between ER-like and INR-like groups was characterized by tumor functional state feature-related signatures; TME-related signatures, including immune and stromal components; and metabolic reprogramming signatures (FIG. 5, panels A and B, Supplementary FIG. 7). Specifically, 29 signatures (including PI3K_AKT_MTOR_SIGNALING, cell cycle, Homologous recombination repair (HRR), DNA damage repair (DDR), Endothelial-to-Mesenchymal transition (EMT), Mismatch repair (MMR), and MTORC1_SIGNALING) were used to describe the functional states of tumor cells. HRR signature (FIG. 5, panel B top pane, p=1.3e-06), PI3K_AKT_MTOR (FIG. 5, panel B top pane, p=0.0055), and DDR signature (Supplementary FIG. 7, p=1.3e-05) had higher GSVA scores in the INR-like group compared to the ER-like group. In comparison, the signature of the ESTROGEN_RESPONSE functional states of tumor cells (Supplementary FIG. 7, p=0.0041) had higher GSVA scores in the ER-like group. We observed marked upregulation of anti-tumor immune components, such as natural killer cells and T regulatory cells (FIG. 5, panel B top pane, p=1.7e-05), suggesting immune escape components were distinctly regulated in the INR-like group. Metabolic reprogramming also differed significantly between the ER-like and INR-like groups. We analyzed the metabolic pathways obtained from the KEGG database and observed that steroid hormone metabolism (FIG. 5, panel B top pane, p=0.00012), and steroid hormone biosynthesis (FIG. 5, panel B top pane, p=0.00095) downregulated in the INR-like group. Unsurprisingly, the 13 CMBs were predictive of many of these signature scores, including HRR (FIG. 5, panel B bottom pane, R=0.29, p=8.5e-09), natural killer cells (FIG. 5, panel B bottom pane, R=0.29, p=3e-09) and DDR (Supplementary FIG. 7, R=0.27, p=8.6e-06). In addition, using these pre-built CMB-signature models from the TCGA-PRAD cohort, we can predict the signature scores in the independent hospital cohort, which show significant differences between the ER-like and INR-like patient groups and are consistent with our observations in the TCGA-PRAD cohort (Supplementary FIG. 8).

INR-Like Patients have More Robust Activation of the mTOR Signaling Pathway and are More Sensitive to mTOR Inhibitor

Applying the pre-built CMB-signature models from the TCGA-PRAD cohort on a subset of patients in the independent cohort (n=20) with matching specimens before and after NADT treatment revealed the impact of NADT treatment on various molecular signatures. Specifically, in both ER-like (n=10) and INR-like (n=10) groups, NADT treatment led to increased NK cells (FIG. 6, panel B; ER-like: p=4.1e-05; INR-like: p=2.8e-6), increased EMT (FIG. 6, panel C; ER-like: p=4.1e-05; INR-like: p=2.8e-6), and defected homologous recombination repair (FIG. 6, panel D; ER-like: p=0.00016; INR-like: p=5.7e-6). Interestingly, the mTOR activity seems not to be affected by NADT treatment (FIG. 6, panel A; ER-like: p=0.67; INR-like: p=0.065), and it remains higher in the INR-like group regardless of NADT treatment. This observation was consistent with the higher protein expression related to mTOR pathway in the TCGA-PRAD INR-like group (FIG. 6, panel E; p-mTOR: p=0.15; p70S6K: p=1.6e-05) and suggested that the INR-like group is significantly more sensitive to mTOR inhibitors (FIG. 6, panel G to FIG. 8, panel H; Rapamycin: p=8.3e-05; Temsirolimus: p=0.57). Notably, the substantially higher mTOR activity in the INR-like group was validated using immunohistochemical (IHC) staining in the independent hospital cohort (FIG. 6, panels I-K, p-mTOR, p=0.063; FIG. 6, panels M-O, p70S6K, p=0.03). The strong and significant correlation between the predicted mTOR signaling score and the protein expression of p-mTOR (FIG. 6, panel L, R=0.66, p=0.0015) and p70S6K (FIG. 6, panel O, R=0.68, p=0.0013) confirmed the predictive power of the prebuilt CMB-signature models.

### Discussion

Intense NADT trials have yielded lower recurrence rates among PCa patients with minimal residual disease after treatment (5-14). Genomic and histologic features associated with NADT treatment resistance at baseline have recently been identified (20); here we developed and validated robust artificial intelligence-powered CMB predictive biomarkers of NADT response. Discovery using whole slide images of needle biopsies from a clinical trial cohort identified 13 CMBs employed in a predictive model, the 13-CMB model. This model was validated in two independent cohorts to accurately predict both response to NADT and prognosis. We also describe the interplay of the CMBs with genetic variants towards NADT resistance, and association with distinct molecular alterations and the TME. Furthermore, we constructed predictive models using 13 CMBs towards various molecular signatures, validated the predictive power and our findings on the mTOR signaling pathway through IHC staining, and revealed potential benefits from mTOR inhibition in patients resistant to NADT. In addition, the predicted changes of specific molecular signatures, including NK cells, EMT and Homologous recombination before and after NADT treatment are consistent with our current understanding of the impact of ADT on DNA repair (35), EMT (36), and immune system response (37). The application of CMBs may provide an accurate method for personalized management of PCa, whereas the association of CMB with specific molecular features may help with developing an understanding of its biology, thus facilitating future drug development.

Current guidelines (3) include clinical and pathologic features, such as PSA level, to define risk classes in prostate cancer. Prior studies have shown clinical factors such as baseline PSA and PSA dynamics and extent of bone involvement as predictors of response to ADT (38). Molecular features such as loss of chromosome 10q (containing PTEN) and TP53 alterations, as well as nuclear ERG expression and the presence of intraductal carcinoma of the prostate have been shown to be predictive of poor outcomes (20). Androgen receptor mutations are frequently seen in patients with metastatic castrate-resistant PCa (39) and prior studies have shown these mutations may even contribute to resistance to androgen receptor signaling inhibitors such as enzalutamide and abiraterone (40). Additional mutations such as ATM, BRCA, and TP53 may confer resistance to these drugs and thus lead to poor outcomes as well (40). Baseline tumor volume estimation using a multi-parametric MRI was predictive of response to NADT as well. The CMB model we describe here was shown to perform better than the described models that incorporated an MRI, presence of intraductal carcinoma, 10q loss, nuclear ERG overexpression, or altered TP53.

Previously, genome-wide association studies have identified >100 common SNPs that may be associated with susceptibility to PCa (41). Genetic polymorphisms associated with the efficacy of ADT have also been described (29). In this study, we reveal the potential interplay between genetic variants and cellular morphometric architecture on NADT treatment response and describe a multimodal signature that combines the four SNP-associated genes and 13 CMBs with an increased power to predict prognosis. This is the first effort where the causal effects between genetic polymorphic changes and cell morphometric architecture on NADT response are described.

Genomic instability (31,32) is associated with localized PCa recurrence and progression to aggressive cancer (33). It is reported that a high tumor mutational burden (TMB) and MSI-high status are predictive of response to immune checkpoint blockade in patients with PCa (42,43). In our analysis, we discover that the majority of CMBs associated with favorable response to NADT treatment are associated with lower TMB and/or MSI. In contrast, the majority of CMBs associated with unfavorable NADT response are related to elevated TMB and/or MSI, suggesting that this group of patients may have a better response if treated additionally with immune checkpoint inhibitors. Thus, certain CMBs could be used in conjunction as biomarkers that predict response to immunotherapy with or without ADT (44-46).

The TME includes immune and non-immune components that have been shown to contribute to disease prognosis (47). In our analyses, we report the association of TME immune elements (CD4+ T cells, regulatory T cells, and mast cells) with a CMB that associates with poor response to ADT. Previous studies have shown mast cells to be pro-tumorigenic (48). There have been conflicting data on the relationship between infiltration of lymphocytes and survival in prostate cancer. However, studies suggest CD4+ regulatory T cells are associated with worse prognosis (49).

In addition to predicting response to ADT, we also show that certain CMBs may be able to predict molecular signatures and the response to drugs targeting specific pathways including the mTOR pathway and DDR pathway. The abnormal regulation of mTOR has been extensively reported within human carcinomas from several different origins (50), and the combination of ADT and mTOR inhibition may be of therapeutic value in PCa treatment (51). We discovered (proteomics in TCGA-PRAD) and validated (IHC in independent hospital cohort) higher mTOR activity in the INR-like group compared to the ER-like group, which is consistent with a previous report of mTOR signaling as a resistance mechanism for PCa ADT therapy (52). Moreover, we revealed higher sensitivity (estimated by IC50 score) to mTOR inhibitor (rapamycin) of the INR-like group compared to the ER-like group, suggesting potential benefit from mTOR targeted therapy for patients in the INR-like group. Although multiple studies have shown no clinical benefit from metformin in prostate cancer (53,54), there was a subgroup of patients with high volume disease in the latter trial, where clinical benefit was described. These findings align with what we describe here in that we see potential benefits from mTOR pathway inhibition in the INR-like group, warranting further evaluation of drugs to block this pathway in this specific subgroup of patients.

DNA-damage repair (DDR) pathway alterations are detected in about 20% of patients with prostate cancer and are associated with improved sensitivity to poly(ADP ribose) polymerases (PARP) inhibitors. Several mechanisms can be activated to repair damaged DNA, one of the most crucial ones being homologous recombination repair (HRR), with the breast cancer genes (BRCA1 and BRCA2) playing a pivotal role. Recently, several clinical trials have reported on the benefit of PARP inhibitors in these patients (55-57). We discovered that DDR signature has higher activity (estimated by GSVA score) in the INR-like group compared to the ER-like group, a potential benefit from DDR targeted therapy for patients in the INR-like group (58-62) and also warrants evaluation in a prospective setting.

Steroid hormones, particularly androgens, play an essential role in both the development of benign prostatic hyperplasia and the stimulation of PCa growth (63), and genes associated with steroid hormones predict the PCa prognosis (64). Upregulation of cholesterol and steroid hormone biosynthesis in PCa cells, driving them into androgen receptor targeted therapy resistance. Blocking both pathways may therefore be a promising approach to overcome resistance to androgen receptor deprivation therapies in PCa (65), which may explain the ADT sensitivity of patients in the ER-like group given its elevated activity in steroid hormone biosynthesis.

This study has several limitations. First, the clinical trial cohort for 13-CMB model development was relatively small. Second, the hospital validation was performed retrospectively with only one center involved. Third, the treatment information in the TCGA-PRAD cohort is incomplete. Nevertheless, our findings on both the 13-CMB model and its underlying molecular association warrant future evaluation in larger multicenter cohorts, and in a prospective clinical trial.

In conclusion, we developed 13-CMB model based on a needle biopsy H&E that may accurately predict NADT treatment response and prognosis in localized PCa. This model shows associations with TME composition, markers of genome instability, and important biological pathways that suggest the underlying molecular mechanisms for its predictive power. We also show the predictive power of the 13-CMB model, including validation of prediction of mTOR activity, where we confirmed the elevated mTOR activity in the INR-like group and revealed the increased sensitivity and potential therapeutic benefit from mTOR inhibitors in the INR-like group. Our study highlights a novel advancement of AI in digital pathology through the unification of robust predictive power with biological interpretability, molecular mechanisms and treatment optimization potentials, which the classic deep learning systems (66) can rarely offer. Thus, it warrants future prospective validation in larger cohorts.

### Methods

### Retrospective Study Design

The study design is illustrated in FIG. 1 (panels A-D). Specifically, the CMB-ML pipeline was applied on WSI of needle biopsy (FIG. 1, panel A) from 37 patients enrolled in a clinical trial (NCT02430480) for CMB identification and model construction (FIG. 1, panel B). The independent validation hospital cohort of 122 localized primary PCa patients at or above intermediate risk according to NCCN risk classification (22) with needle biopsy, complete clinical, pathological, and follow-up information between 2017 and 2022 was retrospectively retrieved from the Nanjing Drum Tower Hospital (FIG. 1, panel C). The entire cohort of 272 patients included 150 cases excluded due to missing clinical and/or pathological data (56 cases), lost to follow-up (61 cases), and insufficient quality of specimens for analysis (33 cases). Patients were followed up through January 2024. The validation study was independently carried out at the Nanjing Drum Tower Hospital, and the study was approved by the Ethics Board of the Nanjing Drum Tower Hospital with a waiver of informed consent. The TCGA-PRAD cohort (FIG. 1, panel D), consisting of 396 H&E-stained diagnostic slides from localized primary PCa patients with matching clinical data, was used to evaluate the prognosis and molecular association of the CMB model.

### CMB Identification and Predictive Model Construction

Based on the stacked predictive sparse decomposition (67) technique and our cellular morphometric biomarker by machine learning (CMB-ML) pipeline (68-70), we defined 256 CMBs from cellular objects extracted from the WSI of H&E stained tissue histology sections from the needle biopsies of 37 patients enrolled in a clinical trial (NCT02430480). In the CMB-ML pipeline, we used a single network layer with 256 dictionary elements (i.e., CMBs) and a sparsity constraint of 30 at a fixed random sampling rate of 1000 cellular objects per WSIs from the cohort. The pre-trained SPSD model reconstructed each cellular region as a sparse combination of pre-defined 256 CMBs and thereafter represents each patient as an aggregation of all delineated cellular objects belonging to the same patient.

The predictive effect of high or low levels of each CMB on NADT treatment response (i.e., INR and ER) was assessed by chi-square test, where the NCT02430480 cohort was divided into two groups (i.e., CMB-high and CMB-low groups) based on each CMB with cut-off point optimized towards minimized p-value during the chi-square test. The set of CMBs with p-value<0.05 was selected as a predictive signature for LASSO (Least Absolute Shrinkage and Selection Operator, glmnet package in R, Version 4.1-4) regression model construction towards NADT treatment response (i.e., INR and ER). The model parameter (i.e., lambda, and coefficients) was optimized using cross-validation (Supplementary FIG. 1, panels A and B), and the cut point at 64.2% on estimated probability was optimized by bootstrapping strategy (80% sampling rate with 100 iterations) on the Youden index (cutpointr package in R, Version 1.1.2).

Exploration of the Underlying Association Between Genetic Variants and CMBs and their Interplay in NADT Treatment Resistance

Mann-Whitney non-parametric test was used to evaluate a significant association between CMBs and 146 risk SNPs related to PCa provided by the NCT02430480 cohort (p<0.05). CMB-SNP-Network was then constructed and visualized in Cytoscape (version 3.8.2). Mediation analysis, a statistical model to determine whether the relationship between two variables (e.g., genetic variant and NADT treatment resistance) is mediated through a third variable (e.g., CMB), was performed using a mediation package (version 4.4.7) and visualized using ggplot2 package (version 3.2.1) in R (version 3.6.0).

### Exploration of the Underlying Association Between Tumor Microenvironments (TMEs) and CMBs in TCGA-PRAD

The TME (i.e., abundances of member cell types in a mixed cell population) was assessed using ConsensusTME (version: 0.0.1.9000) (71). The association between CMBs and TMEs was calculated by Spearman rank correlation, and represented by a heatmap (ComplexHeatmap package in R, version 3.18).

### Exploration of the Underlying Association Between Genomic Instability and CMBs in TCGA-PRAD

Genomic instability in terms of aneuploidy score and fraction of genome altered, mutation counts, mutation burden, and prognosis (i.e., overall survival and progression-free survival) were downloaded from cBioPortal, and the association between CMBs and genomic instability parameters was calculated by Spearman correlation, and represented by a heatmap (ComplexHeatmap package in R, version 3.18).

### Exploration of Underlying Molecular Associations of CMB in TCGA-PRAD

The CMB-Enrichment-Network study was performed based on the following steps: (1) significantly CMB-associated genes were selected per cancer type per CMB (Spearman correlation, |correlation coefficient|>0.20 and p<0.05, R version 3.6.0); (2) Enrichment analysis (i.e., BP/Biological Process, CC/Cellular Component, MF/Molecular Function, and KEGG/Kyoto Encyclopedia of Genes and Genomes) was performed (clusterProfiler package in R, version 4.1.0) on CMB-associated genes per CMB; and (3) CMB-BP-Network, CMB-CC-Network, CMB-MF-Network and CMB-KEGG network were then constructed and visualized in Cytoscape (version 3.8.2).

### Evaluation of the TME-Related Signature Scores in TCGA-PRAD Patients

After reviewing previously published studies, the Molecular Signatures Database (MSigDB; webpage for: gsea-msigdb.org/gsea/msigdb/index.jsp), and the Reactome pathway portal (webpage for: reactome.org/PathwayBrowser/), we identified relevant biomarker genes for tumor, immune, stromal, and metabolic reprogramming signatures. The 61 TME-related signature as well as the source of each signature were included in this study. The signature score of each TME-related signature was calculated per sample using gene set variation analysis (GSVA) (‘GSVA’ package, version 1.46.0). Except for particular indication, the visualization of heatmap was achieved by using the R package ggplot2 (version 3.4.1).

### Evaluation of Other Biological Pathway Enrichment Scores in TCGA-PRAD Patients

Human metabolism-related pathways were obtained from the KEGG database (webpage for: genome.jp/kegg/). A previously published study retrieved 86 human metabolism-related pathways and ten oncogenic signatures containing an HR signature. GSVA was performed to calculate the enrichment score of each signature for each sample. To identify the potential differences in the biological functions of genes among high and low-risk groups, GSEA was performed based on the gene signatures using the R package ‘clusterprofiler’ (version 4.6.2).

### Construction and Evaluation of CMB-Signature Models for the Prediction of Molecular Signature Scores in TCGA-PRAD and Independent Hospital Cohort

Random forest regression model (randomForest package in R, Version 4.6-14) and 5-fold cross-validation strategy were deployed to assess the predictive power of CMBs towards the molecular signatures on hold-out samples (i.e., the samples were not used during training) in TCGA-PRAD patients, and therefore constructed five models per signature. Spearman correlation was used to evaluate the performance of predicted scores compared to the ground truth (i.e., scores estimated by GSVA). During the CMB-Signature model application in an independent hospital cohort, the predicted score, for a specific signature, was defined as an average score from all five models corresponding to this signature.

### Cellular Morphometric Biomarker Risk Score (CMBRS) Construction and Patient Stratification in the TCGA-PRAD Cohort

The construction of the cellular morphometric biomarker risk score (CMBRS) in the TCGA-PRAD cohort is defined below. The coefficients of the final CMBs as categorical variables were obtained from multivariate CoxPH regression analysis on PFS:

$$CMBRS = {\sum }_{i = 1}^{N} ( coefficient of {CMB\_Category}^{i} ) * ( {CMB\_Category}^{i} )$$

In the above equation, N is the number of final predictive CMBs that were pre-selected from NCT02430480 cohort, and CMB_Categoryⁱ is the category of the i^{th} CMB (i.e., CMB-high=1; CMB-low=0) where TCGA-PRAD cohort was divided into CMB-high/-low groups based on each CMB (cut-off estimated using survminer package in R, version 0.4.8, based on PFS). Then, we stratified the TCGA-PRAD cohort into three groups (High: top third; Intermediate: middle third; and Low: bottom third) based on CMBRS.

### Gene Risk Score (GERS) Construction and Patient Stratification in TCGA-PRAD Cohort

The construction of the GERS in the TCGA-PRAD cohort is defined below. The coefficients of the genes as categorical variables were obtained from multivariate CoxPH regression analysis on PFS:

$$GERS = {\sum }_{i = 1}^{N} ( coefficient of {Gene\_Category}^{i} ) * ( {Gene\_Category}^{i} )$$

In the above equation, N is the number of genes that were pre-selected from NCT02430480 cohort that associated with specific SNPs, and Gene_Categoryⁱ is the category of the i^{th} gene (i.e., Gene-high=1; Gene-low=0) where TCGA-PRAD cohort was divided into Gene-high/-low groups based on each gene (cut-off estimated using survminer package in R, version 0.4.8, based on PFS). Then, we stratified the TCGA-PRAD cohort into three groups (High: top third; Intermediate: middle third; and Low: bottom third) based on GERS.

### Cellular Morphometric Biomarker and Gene Risk Score (CMBGERS) Construction and Patient Stratification in TCGA-PRAD Cohort

Construction of the cellular morphometric biomarker and gene risk score (CMBGERS) in the TCGA-PRAD cohort is defined below. The coefficients of the CMBRS and GERS were obtained from multivariate CoxPH regression analysis on PFS:

$$CMBGERS = coefficient of CMBRS * CMBRS + coefficient of GERS * GERS$$

Then, we stratified the TCGA-PRAD cohort into three groups (High: top third; Intermediate: middle third; and Low: bottom third) based on CMBGERS.

### Immunohistochemical (IHC) Staining

IHC staining was carried out on 4-μm sections of formalin-fixed and paraffin-embedded tissues according to the standard protocol on a subset of the independent hospital cohort (20 patients in total; ten patients from the ER-like group, and ten patients from the INR-like group). The selection criteria are: (1) availability of sufficient specimens from needle biopsy before NADT for IHC staining; (2) availability of matching diagnostic slides from prostatectomy after NADT; and (3) balanced number of patients in ER-like and INR-like groups that meets the requirements (1) and (2). During IHC staining, sections were dewaxed and rehydrated in serial alcohol washes, and then endogenous peroxidase activities were blocked. After the nonspecific sites were saturated with 5% normal goat serum, the sections were incubated overnight at 4° C. with anti-phospho-mTOR (1:50, mouse mAb, #67778-1-Ig, Proteintech) and anti-phospho-p70s6k (1:50, rabbit pAb, #ab2571, Abcam), and then incubated with anti-rabbit or anti-mouse Ig secondary antibodies. The sections were visualized using the biotin-peroxidase complex and counterstained with hematoxylin.

To assess p-mTOR and p-p70s6k, the stained sections were screened at low-power magnification (×20), and five hot spots were selected. The expression levels of p-mTOR and p-p70s6k were quantified using the H-score method (72), which was a semi-quantitative assessment combining both staining intensity (0: no staining; 1: weak staining; 2: moderate staining; and 3: intense staining) and percentage of positive cells with a numerical range from 0 to 300.

### Statistical Analysis

All analysis was performed with R (Version 4.0.2). A predictive model based on CMB signature was constructed using logistic regression in R. Predictive power was assessed by accuracy, sensitivity, specificity, and area under the ROC Curve (AUC, pROC package in R, version 1.18.0). Survival differences between subtypes or groups were examined by log-rank test. Differences concerning the expression of immune checkpoints, immune cell infiltration, and genomic instability between groups were analyzed with the Mann-Whitney non-parametric test (for continuous variables) or Chi-square test (for categorical variables). P value (FDR corrected if applicable)<0.05 was considered statistically significant.

### Data Availability

Whole slide images and clinical data of the NCT02430480 cohort were downloaded from the Cancer Imaging Archive (TCIA, webpage for: doi.org/10.7937/TCIA.JHQD-FR46). Clinical and metadata were acquired from the original publication of the NCT02430480 study. Whole slide images of the TCGA-PRAD cohort were downloaded from the TCGA GDC portal (webpage for: portal.gdc.cancer.gov/). Clinical and molecular data were downloaded from cBioportal (webpage for: cbioportal.org/). All NCT02430480 and TCGA data were publicly available without modification. Raw data from the Nanjing Drum Tower Hospital is not currently permitted in public repositories because ethical and legal implications are still being discussed at an institutional level.

### REFERENCES CITED HEREIN

1. Siegel R L, Miller K D, Wagle N S, Jemal A. Cancer statistics, 2023. CA: A Cancer Journal for Clinicians 2023; 73(1):17-48.
2. Institute NC. 2020 Cancer Stat Facts: Prostate Cancer.
3. Schaeffer E M, Srinivas S, Adra N, An Y, Barocas D, Bitting R, et al. Prostate Cancer, Version 4.2023, NCCN Clinical Practice Guidelines in Oncology. J Natl Compr Canc Netw 2023; 21(10):1067-96 doi 10.6004/jnccn.2023.0050.
4. Rebello R J, Oing C, Knudsen K E, Loeb S, Johnson D C, Reiter R E, et al. Prostate cancer. Nature Reviews Disease Primers 2021; 7(1):9 doi 10.1038/s41572-020-00243-0.
5. McKay R R, Ye H, Xie W, Lis R, Calagua C, Zhang Z, et al. Evaluation of Intense Androgen Deprivation Before Prostatectomy: A Randomized Phase II Trial of Enzalutamide and Leuprolide With or Without Abiraterone. Journal of Clinical Oncology 2019; 37(11):923-31 doi 10.1200/jco.18.01777.
6. Lee L S, Sim A Y L, Ong C W, Yang X, Ng C C Y, Liu W, et al. NEAR trial: A single-arm phase II trial of neoadjuvant apalutamide monotherapy and radical prostatectomy in intermediate- and high-risk prostate cancer. Prostate Cancer and Prostatic Diseases 2022; 25(4):741-8 doi 10.1038/s41391-022-00496-8.
7. Devos G, Tosco L, Baldewijns M, Gevaert T, Goffin K, Petit V, et al. ARNEO: A Randomized Phase II Trial of Neoadjuvant Degarelix with or Without Apalutamide Prior to Radical Prostatectomy for High-risk Prostate Cancer. European Urology 2023; 83(6):508-18.
8. Efstathiou E, Boukovala M A, Spetsieris N, Wen S, Hoang A, Weldon J A, et al. Neoadjuvant apalutamide (APA) plus leuprolide (LHRHa) with or without abiraterone (AA) in localized high-risk prostate cancer (LHRPC). Journal of Clinical Oncology 2020; 38(15_suppl):5504—doi 10.1200/JCO.2020.38.15_suppl.5504.
9. Karzai F, Walker S M, Wilkinson S, Madan R A, Shih J H, Merino M J, et al. Sequential Prostate Magnetic Resonance Imaging in Newly Diagnosed High-risk Prostate Cancer Treated with Neoadjuvant Enzalutamide is Predictive of Therapeutic Response. Clinical Cancer Research 2021; 27(2):429-37 doi 10.1158/1078-0432.Ccr-20-2344.
10. Efstathiou E, Davis J W, Pisters L, Li W, Wen S, McMullin R P, et al. Clinical and Biological Characterisation of Localised High-risk Prostate Cancer: Results of a Randomised Preoperative Study of a Luteinising Hormone-releasing Hormone Agonist with or Without Abiraterone Acetate plus Prednisone. European Urology 2019; 76(4):418-24.
11. Montgomery B, Tretiakova M S, Joshua A M, Gleave M E, Fleshner N, Bubley G J, et al. Neoadjuvant Enzalutamide Prior to Prostatectomy. Clinical Cancer Research 2017; 23(9):2169-76 doi 10.1158/1078-0432.Ccr-16-1357.
12. Davis J, Chapin B, Pettaway C, Chery L, Pisters L, Ward J, et al. PD61-11 A PHASE II STUDY OF 6-MONTHS APALUTAMIDE PRIOR TO RADICAL PROSTATECTOMY IN INTERMEDIATE RISK PATIENTS TO REDUCE THE FREQUENCY OF PATHOLOGIC FEATURES THAT DRIVE POST-OPERATIVE RADIATION THERAPY. Journal of Urology 2021; 206 doi 10.1097/JU.0000000000002098.11.
13. McKay R R, Xie W, Ye H, Fennessy F M, Zhang Z, Lis R, et al. Results of a Randomized Phase II Trial of Intense Androgen Deprivation Therapy prior to Radical Prostatectomy in Men with High-Risk Localized Prostate Cancer. J Urol 2021; 206(1):80-7 doi 10.1097/ju.0000000000001702.
14. Ilario E N, Bastos D A, Guglielmetti G B, Murta C B, Cardili L, Cordeiro M D, et al. Perioperative Morbidity of Radical Prostatectomy After Intensive Neoadjuvant Androgen Blockade in Men With High-Risk Prostate Cancer: Results of Phase II Trial Compared to a Control Group. Clinical Genitourinary Cancer 2023; 21(1):43-54 doi 10.1016/j.clgc.2022.10.009.
15. MacLennan S, Azevedo N, Duncan E, Dunsmore J, Fullwood L, Lumen N, et al. Mapping European Association of Urology Guideline Practice Across Europe: An Audit of Androgen Deprivation Therapy Use Before Prostate Cancer Surgery in 6598 Cases in 187 Hospitals Across 31 European Countries. European Urology 2023.
16. Taplin M E, Montgomery B, Logothetis C J, Bubley G J, Richie J P, Dalkin B L, et al. Intense androgen-deprivation therapy with abiraterone acetate plus leuprolide acetate in patients with localized high-risk prostate cancer: results of a randomized phase II neoadjuvant study. J Clin Oncol 2014; 32(33):3705-15 doi 10.1200/jco.2013.53.4578.
17. Montgomery B, Tretiakova M S, Joshua A M, Gleave M E, Fleshner N, Bubley G J, et al. Neoadjuvant Enzalutamide Prior to Prostatectomy. Clin Cancer Res 2017; 23(9):2169-76 doi 10.1158/1078-0432.Ccr-16-1357.
18. McKay R R, Ye H, Xie W, Lis R, Calagua C, Zhang Z, et al. Evaluation of Intense Androgen Deprivation Before Prostatectomy: A Randomized Phase II Trial of Enzalutamide and Leuprolide With or Without Abiraterone. J Clin Oncol 2019; 37(11):923-31 doi 10.1200/jco.18.01777.
19. Liu X-P, Jin X, Seyed Ahmadian S, Yang X, Tian S-F, Cai Y-X, et al. Clinical significance and molecular annotation of cellular morphometric subtypes in lower-grade gliomas discovered by machine learning. Neuro-Oncology 2022:noac154 doi 10.1093/neuonc/noac154.
20. Wilkinson S, Ye H, Karzai F, Harmon S A, Terrigino N T, VanderWeele D J, et al. Nascent Prostate Cancer Heterogeneity Drives Evolution and Resistance to Intense Hormonal Therapy. Eur Urol 2021; 80(6):746-57 doi 10.1016/j.eururo.2021.03.009.
21. Clark K, Vendt B, Smith K, Freymann J, Kirby J, Koppel P, et al. The Cancer Imaging Archive (TCIA): Maintaining and Operating a Public Information Repository. Journal of Digital Imaging 2013; 26(6):1045-57 doi 10.1007/s10278-013-9622-7.
22. Society A C. Prostate cancer risk groups.
23. Liu X-P, Jin X, Seyed Ahmadian S, Yang X, Tian S-F, Cai Y-X, et al. Clinical significance and molecular annotation of cellular morphometric subtypes in lower-grade gliomas discovered by machine learning. Neuro-Oncology 2022; 25(1):68-81 doi 10.1093/neuonc/noac154.
24. Perez-Baena M J, Mao J H, Perez-Losada J, Santos-Briz A, Chang H, Canueto J. AI-empowered cellular morphometric risk score improves prognostic stratification of cutaneous squamous cell carcinoma. Clin Exp Dermatol 2023 doi 10.1093/ced/llad264.
25. Mao X, Cai Y, Long S, Perez-Losada J, Mao J H, Chang H. Pan-cancer evaluation of clinical value of mitotic network activity index (MNAI) and its predictive value for immunotherapy. Front Oncol 2023; 13:1178568 doi 10.3389/fonc.2023.1178568.
26. Chang H, Yang X, Moore J, Liu X-P, Jen K-Y, Snijders A M, et al. From Mouse to Human: Cellular Morphometric Subtype Learned From Mouse Mammary Tumors Provides Prognostic Value in Human Breast Cancer. Frontiers in Oncology 2022; 11 doi 10.3389/fonc.2021.819565.
27. Mermel C H, Schumacher S E, Hill B, Meyerson M L, Beroukhim R, Getz G. GISTIC2.0 facilitates sensitive and confident localization of the targets of focal somatic copy-number alteration in human cancers. Genome Biology 2011; 12(4):R41 doi 10.1186/gb-2011-12-4-r41.
28. Wang A, Shen J, Rodriguez A A, Saunders E J, Chen F, Janivara R, et al. Characterizing prostate cancer risk through multi-ancestry genome-wide discovery of 187 novel risk variants. Nat Genet 2023; 55(12):2065-74 doi 10.1038/s41588-023-01534-4.
29. Ross R W, Oh W K, Xie W, Pomerantz M, Nakabayashi M, Sartor O, et al. Inherited Variation in the Androgen Pathway Is Associated With the Efficacy of Androgen-Deprivation Therapy in Men With Prostate Cancer. Journal of Clinical Oncology 2008; 26(6):842-7 doi 10.1200/jco.2007.13.6804.
30. Ge R, Wang Z, Cheng L. Tumor microenvironment heterogeneity an important mediator of prostate cancer progression and therapeutic resistance. npj Precision Oncology 2022; 6(1):31 doi 10.1038/s41698-022-00272-w.
31. Boutros P C, Fraser M, Harding N J, de Borja R, Trudel D, Lalonde E, et al. Spatial genomic heterogeneity within localized, multifocal prostate cancer. Nat Genet 2015; 47(7):736-45 doi 10.1038/ng.3315.
32. Abeshouse A, Ahn J, Akbani R, Ally A, Amin S, Andry Christopher D, et al. The Molecular Taxonomy of Primary Prostate Cancer. Cell 2015; 163(4):1011-25 doi 10.1016/j.cell.2015.10.025.
33. Hieronymus H, Murali R, Tin A, Yadav K, Abida W, Moller H, et al. Tumor copy number alteration burden is a pan-cancer prognostic factor associated with recurrence and death. Elife 2018; 7 doi 10.7554/eLife.37294.
34. Azam H, Maher S, Clarke S, Gallagher W M, Prencipe M. SRF inhibitors reduce prostate cancer cell proliferation through cell cycle arrest in an isogenic model of castrate-resistant prostate cancer. Cell Cycle 2023; 22(14-16):1759-76 doi 10.1080/15384101.2023.2229713.
35. Polkinghorn W R, Parker J S, Lee M X, Kass E M, Spratt D E, Iaquinta P J, et al. Androgen receptor signaling regulates DNA repair in prostate cancers. Cancer Discov 2013; 3(11):1245-53 doi 10.1158/2159-8290.Cd-13-0172.
36. Niu Y J. AB17. ADT treatment promotes PCa EMT and metastasis. Transl Androl Urol. 2014 September; 3(Suppl 1):AB17. doi: 10.3978/j.issn.2223-4683.2014.s017.
37. Schwermann M, Huntington K E, Carlsen L, Zhou L, Srinivasan P, George A, et al. Abstract 1843: Androgen signaling blockade enhances NK cell-mediated killing of prostate cancer cells (PC) and promotes NK cell tumor infiltration in vivo. Cancer Research 2023; 83(7_Supplement):1843—doi 10.1158/1538-7445.Am2023-1843.
38. Divrik R T, Türkeri L, S̨ahin A F, Akdoǧan B, Ates̨ F, C̨al C̨, et al. Prediction of response to androgen deprivation therapy and castration resistance in primary metastatic prostate cancer. Urol Int 2012; 88(1):25-33 doi 10.1159/000334539.
39. Grasso C S, Wu Y M, Robinson D R, Cao X, Dhanasekaran S M, Khan A P, et al. The mutational landscape of lethal castration-resistant prostate cancer. Nature 2012; 487(7406):239-43 doi 10.1038/nature11125.
40. Annala M, Vandekerkhove G, Khalaf D, Taavitsainen S, Beja K, Warner E W, et al. Circulating Tumor DNA Genomics Correlate with Resistance to Abiraterone and Enzalutamide in Prostate Cancer. Cancer Discov 2018; 8(4):444-57 doi 10.1158/2159-8290.Cd-17-0937.
41. Benafif S, Kote-Jarai Z, Eeles R A. A Review of Prostate Cancer Genome-Wide Association Studies (GWAS). Cancer Epidemiol Biomarkers Prev 2018; 27(8):845-57 doi 10.1158/1055-9965.Epi-16-1046.
42. Abida W, Cheng M L, Armenia J, Middha S, Autio K A, Vargas H A, et al. Analysis of the Prevalence of Microsatellite Instability in Prostate Cancer and Response to Immune Checkpoint Blockade. JAMA Oncol 2019; 5(4):471-8 doi 10.1001/jamaoncol.2018.5801.
43. Sayegh N, Graf R, Fisher V, Weberpals J, Huang R S P, Lin D I, et al. Tumor mutational burden as a predictive biomarker for immune checkpoint inhibitor versus taxane chemotherapy benefit in metastatic castration-resistant prostate cancer: A real-world biomarker study. Journal of Clinical Oncology 2022; 40(6_suppl):162—doi 10.1200/JCO.2022.40.6_suppl.162.
44. Kim J E, Lee K, Kim I Y. Current Update on Prostate Cancer Immunotherapy. J Urol Oncol 2023; 21(1):14-22 doi 10.22465/juo.234600100005.
45. Qin C, Wang J, Du Y, Xu T. Immunosuppressive environment in response to androgen deprivation treatment in prostate cancer. Front Endocrinol (Lausanne) 2022; 13:1055826 doi 10.3389/fendo.2022.1055826.
46. Hawley J E, Obradovic A Z, Dallos M C, Lim E A, Runcie K, Ager C R, et al. Anti-PD-1 immunotherapy with androgen deprivation therapy induces robust immune infiltration in metastatic castration-sensitive prostate cancer. Cancer Cell 2023; 41(11):1972-88.e5 doi 10.1016/j.ccell.2023.10.006.
47. Shiao S L, Chu G C, Chung L W. Regulation of prostate cancer progression by the tumor microenvironment. Cancer Lett 2016; 380(1):340-8 doi 10.1016/j.canlet.2015.12.022.
48. Johansson A, Rudolfsson S, Hammarsten P, Halin S, Pietras K, Jones J, et al. Mast cells are novel independent prognostic markers in prostate cancer and represent a target for therapy. Am J Pathol 2010; 177(2):1031-41 doi 10.2353/ajpath.2010.100070.
49. Davidsson S, Ohlson A L, Andersson S O, Fall K, Meisner A, Fiorentino M, et al. CD4 helper T cells, CD8 cytotoxic T cells, and FOXP3(+) regulatory T cells with respect to lethal prostate cancer. Mod Pathol 2013; 26(3):448-55 doi 10.1038/modpathol.2012.164.
50. Hua H, Kong Q, Zhang H, Wang J, Luo T, Jiang Y. Targeting mTOR for cancer therapy. J Hematol Oncol 2019; 12(1):71 doi 10.1186/s13045-019-0754-1.
51. Wang Y, Mikhailova M, Bose S, Pan C X, deVere White R W, Ghosh P M. Regulation of androgen receptor transcriptional activity by rapamycin in prostate cancer cell proliferation and survival. Oncogene 2008; 27(56):7106-17 doi 10.1038/onc.2008.318.
52. Pungsrinont T, Kallenbach J, Baniahmad A. Role of PI3K-AKT-mTOR Pathway as a Pro-Survival Signaling and Resistance-Mediating Mechanism to Therapy of Prostate Cancer. Int J Mol Sci 2021; 22(20) doi 10.3390/ijms222011088.
53. Fleshner N E, Bernardino R M, Lajkosz K, Saad F, Izawa J, Drachenberg D, et al. A randomized, double-blind, placebo-controlled trial of metformin in reducing progression among men on expectant management for low-risk prostate cancer: The MAST (Metformin Active Surveillance Trial) study. Journal of Clinical Oncology 2024; 42(17_suppl):LBA5002-LBA doi 10.1200/JCO.2024.42.17_suppl.LBA5002.
54. Gillessen S, Murphy L R, James N D, Sachdeva A, Attard G, Jones R J, et al. LBA70 Adding metformin to androgen deprivation therapy (ADT) for patients (pts) with metastatic hormone sensitive prostate cancer (mHSPC): Overall survival (OS) results from the multi-arm, multi-stage randomised platform trial STAMPEDE. Annals of Oncology 2024; 35:51258-59 doi 10.1016/j.annonc.2024.08.2313.
55. Hussain M, Mateo J, Fizazi K, Saad F, Shore N, Sandhu S, et al. Survival with Olaparib in Metastatic Castration-Resistant Prostate Cancer. New England Journal of Medicine 2020; 383(24):2345-57 doi doi:10.1056/NEJMoa2022485.
56. Fizazi K, Piulats J M, Reaume M N, Ostler P, McDermott R, Gingerich J R, et al. Rucaparib or Physician's Choice in Metastatic Prostate Cancer. New England Journal of Medicine 2023; 388(8):719-32 doi doi:10.1056/NEJMoa2214676.
57. de Bono J S, Mehra N, Scagliotti G V, Castro E, Dorff T, Stirling A, et al. Talazoparib monotherapy in metastatic castration-resistant prostate cancer with DNA repair alterations (TALAPRO-1): an open-label, phase 2 trial. The Lancet Oncology 2021; 22(9):1250-64 doi 10.1016/S1470-2045(21)00376-4.
58. Shou M, Zhou H, Ma L. New advances in cancer therapy targeting TGF-&#x3b2; signaling pathways. Molecular Therapy—Oncolytics 2023; 31 doi 10.1016/j.omto.2023.100755.
59. Liu S, Ren J, ten Dijke P. Targeting TGFβ signal transduction for cancer therapy. Signal Transduction and Targeted Therapy 2021; 6(1):8 doi 10.1038/s41392-020-00436-9.
60. Barcellos-Hoff M H, Gulley J L. Molecular Pathways and Mechanisms of TGFβ in Cancer Therapy. Clinical Cancer Research 2023; 29(11):2025-33 doi 10.1158/1078-0432.Ccr-21-3750.
61. Liu Q, Chen G, Moore J, Guix I, Placantonakis D, Barcellos-Hoff M H. Exploiting Canonical TGFβ Signaling in Cancer Treatment. Molecular Cancer Therapeutics 2022; 21(1):16-24 doi 10.1158/1535-7163.Mct-20-0891.
62. Guix I, Liu Q, Pujana M A, Ha P, Piulats J, Linares I, et al. Validation of Anticorrelated TGFβ Signaling and Alternative End-Joining DNA Repair Signatures that Predict Response to Genotoxic Cancer Therapy. Clinical Cancer Research 2022; 28(7):1372-82 doi 10.1158/1078-0432.Ccr-21-2846.
63. Bosland M C. The role of steroid hormones in prostate carcinogenesis. J Natl Cancer Inst Monogr 2000(27):39-66 doi 10.1093/oxfordjournals.jncimonographs.a024244.
64. Lai Y-L, Liu C-H, Wang S-C, Huang S-P, Cho Y-C, Bao B-Y, et al. Identification of a Steroid Hormone-Associated Gene Signature Predicting the Prognosis of Prostate Cancer through an Integrative Bioinformatics Analysis. Cancers 2022; 14(6):1565.
65. Neuwirt H, Bouchal J, Kharaishvili G, Ploner C, Jöhrer K, Pitterl F, et al. Cancer-associated fibroblasts promote prostate tumor growth and progression through upregulation of cholesterol and steroid biosynthesis. Cell Commun Signal 2020; 18(1):11 doi 10.1186/s12964-019-0505-5.
66. Spratt D E, Tang S, Sun Y, Huang H C, Chen E, Mohamad O, et al. Artificial Intelligence Predictive Model for Hormone Therapy Use in Prostate Cancer. NEJM Evid 2023; 2(8):EVIDoa2300023 doi 10.1056/EVIDoa2300023.
67. Chang H, Zhou Y, Borowsky A, Barner K, Spellman P, Parvin B. Stacked Predictive Sparse Decomposition for Classification of Histology Sections. Int J Comput Vis 2015; 113(1):3-18 doi 10.1007/s11263-014-0790-9.
68. Liu X P, Jin X, Ahmadian S S, Yang X, Tian S F, Cai Y X, et al. Clinical Significance and Molecular Annotation of Cellular Morphometric Subtypes in Lower Grade Gliomas discovered by Machine Learning. Neuro Oncol 2022 doi 10.1093/neuonc/noac154.
69. Chang H, Yang X, Moore J, Liu X P, Jen K Y, Snijders A M, et al. From Mouse to Human: Cellular Morphometric Subtype Learned From Mouse Mammary Tumors Provides Prognostic Value in Human Breast Cancer. Front Oncol 2021; 11:819565 doi 10.3389/fonc.2021.819565.
70. Mao X Y, Perez-Losada J, Abad M, Rodriguez-Gonzalez M, Rodriguez C A, Mao J H, et al. iCEMIGE: Integration of CEll-morphometrics, MIcrobiome, and GEne biomarker signatures for risk stratification in breast cancers. World J Clin Oncol 2022; 13(7):616-29 doi 10.5306/wjco.v13.i7.616.
71. Jiménez-Sánchez A, Cast O, Miller M L. Comprehensive Benchmarking and Integration of Tumor Microenvironment Cell Estimation Methods. Cancer Res 2019; 79(24):6238-46 doi
10.1158/0008-5472.Can-18-3560.
72. McCarty K S, Jr., Miller L S, Cox E B, Konrath J, McCarty K S, Sr. Estrogen receptor analyses. Correlation of biochemical and immunohistochemical methods using monoclonal antireceptor antibodies. Arch Pathol Lab Med 1985; 109(8):716-21.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method for predicting whether a subject with prostate cancer will be responsive to neoadjuvant therapy (NT), or whether NT will be efficacious for treating a subject with prostate cancer, comprising: (a) obtaining a prostate cancer tumor sample from a subject suffering from prostate cancer, or suspected thereof, (b) determining or measuring the value or quantity of one or more cellular morphometric biomarkers (CMB); and (c) correlating the value or quantity determined or measured for the CMBs to determine whether the subject would benefit from a neoadjuvant androgen deprivation therapy (NT).

2. The method of claim 1, further comprising: (d) treating the subject with NT.

3. The method of claim 2, wherein the treating the subject with NT step (d) comprises treating the subject with a neoadjuvant androgen deprivation therapy (NADT).

4. The method of claim 3, wherein the neoadjuvant androgen deprivation therapy (NADT) comprising administering an androgen receptor blocker to the subject.

5. The method of claim 3, wherein the treating the subject with NT step (d) comprises administering an mTOR inhibitor to the subject, or a DNA damage response (DDR) inhibitor to the subject.

6. A method for predicting whether a subject with prostate cancer will be responsive to neoadjuvant therapy (NT), or whether NT will be efficacious for treating a subject with prostate cancer, comprising: (a) obtaining a prostate cancer tumor sample from a subject suffering from prostate cancer, or suspected thereof, (b) determining or measuring the value or quantity of one or more cellular morphometric biomarkers (CMB); (c) calculating a Cellular Morphometric Biomarker Risk Score (CMBRS) using the CMBs determined or measured in step (b) using the formula:
$$CMBRS = {Σ}_{i = 1}^{N} ( coefficient of {CMB\_Category}^{i} ) * ( {CMB\_Category}^{i} ) ;$$
and
(d) correlating the CMRS to whether the subject would benefit from a neoadjuvant therapy (NT).

7. The method of claim 6, wherein the CMBRS is calculated using 13-CMB model comprising using the following CMBs: CMB 13, CMB 16, CMB 78, CMB 79, CMB 108, CMB 118, CMB 132, CMB 141, CMB 192, CMB 201, CMB 212, CMB 216, and CMB 236.

8. The method of claim 6, further comprising: (d) treating the subject with NT.

9. The method of claim 8, wherein the treating the subject with NT step (d) comprises treating the subject with a neoadjuvant androgen deprivation therapy (NADT).

10. The method of claim 9, wherein the neoadjuvant androgen deprivation therapy (NADT) comprising administering an androgen receptor blocker to the subject. In some embodiments.

11. The method of claim 8, wherein the treating the subject with NT step (d) comprises administering an mTOR inhibitor to the subject, or a DNA damage response (DDR) inhibitor to the subject.
